(19) United States
(12) Patent Application Publication
(10) Pub. No.: US 2004/0063191 A1
(43) Pub. Date: Apr. 1, 2004

(54) Compositions and methods for determining the replication capacity of a pathogenic virus

(76) Inventors: Wei Huang, Foster City, CA (US); Mary T. Wrin, Fremont, CA (US); Andrea Garmanik, Chandler, AZ (US); Jeff Beauchaine, Daly City, CA (US); Jeannette M. Whitcomb, San Mateo, CA (US); Christos J. Petropoulos, Half Moon Bay, CA (US); Neil T. Parkin, Belmont, CA (US)

Correspondence Address: MORGAN, LEWIS & BOCKIUS, LLP., 3300 HILLVIEW AVENUE, PALO ALTO, CA, 94304, US

(21) Appl. No.: 10/612,604

(22) Filed: Jul. 1, 2003

Related U.S. Application Data
(60) Provisional application No. 60/393,306, filed on Jul. 1, 2002.

(51) Int. Cl. C12N 1/20

(52) U.S. Cl. 435/252.3

(57) ABSTRACT

This invention relates to compositions and methods for determining the replication capacity of a non-nucleoside reverse transcriptase inhibitor resistant virus. The compositions and methods are useful for identifying effective drug regimens for the treatment of viral infections, and identifying and determining the biological effectiveness of potential therapeutic compounds.

This application is entitled to and claims priority to U.S. Provisional Application No. 60/393,306, filed Jul. 1, 2002, the contents of which is hereby incorporated by reference in its entirety.

### 1. FIELD OF INVENTION

This invention relates to compositions and methods for determining the replication capacity of a virus. The compositions and methods are useful for identifying effective drug regimens for the treatment of viral infections, and identifying and determining the biological effectiveness of potential therapeutic compounds.

### 2. BACKGROUND OF THE INVENTION

More than 60 million people have been infected with the human immunodeficiency virus (“HIV”), the causative agent of acquired immune deficiency syndrome (“AIDS”), since the early 1980s. See Lucas, 2002, Lepr Rev. 73(1) 64-71. HIV/AIDS is now the leading cause of death in sub-Saharan Africa, and is the fourth biggest killer worldwide. At the end of 2001, an estimated 40 million people were living with HIV globally. See Norris, 2002, Radiol Technol. 73(4) 339-363.

Modern anti-HIV drugs target different stages of the HIV life cycle and a variety of enzymes essential for HIV's replication and/or survival. Amongst the drugs that have so far been approved for AIDS therapy are nucleoside reverse transcriptase inhibitors (“NRTI”) such as AZT, ddI, ddC, d4T, 3TC, abacavir, nucleotide reverse transcriptase inhibitors such as tenofovir, non-nucleoside reverse transcriptase inhibitors (“NNRTI”) such as nevirapine, efavirenz, delavirdine and protease inhibitors such as saquinavir, ritonavir, indinavir, nelfinavir, amprenavir and lopinavir.

One consequence of the action of an anti-viral drug is that it can exert sufficient selective pressure on virus replication to select for drug-resistant mutants (Herrmann et al., 1977, Ann NY Acad Sci 284:632-637). With increasing drug exposure, the selective pressure on the replicating virus population increases to promote the more rapid emergence of drug resistant mutants. Many protease inhibitor resistance mutations and some NRTI resistance mutations are known to impair HIV-1 replication capacity to varying degrees. Typically, mutations conferring resistance to an antiviral drug reduce the replication capacity of the mutant virus. See, e.g., Nijhuis et al., 2001, Curr Op Infect Diseases 14:23-28, incorporated herein by reference in its entirety. Changes in replication capacity of a virus are of major clinical importance because they can affect the response of a patient to anti-viral therapies. See id. However, the effects of NNRTI resistance mutations are largely uncharacterized and it is often assumed that NNRTI resistance is not associated with impaired viral replication. Thus, there is a need in the art for methods and compositions for determining the replication capacity of a NNRTI resistant virus.

### 3. SUMMARY OF THE INVENTION

The present invention provides methods and compositions for determining the replication capacity (also called the replication fitness) of a virus, for example, HIV, e.g., a non-nucleoside reverse transcriptase inhibitor (NNRTI) resistant HIV. The methods and compositions are based on an analysis of a panel of recombinant virus vectors created using site-directed mutagenesis containing one or more reverse transcriptase (RT) amino acid substitutions. The methods and compositions of the invention significantly improve the quality of life of a patient by providing information to the clinician useful for the design of more effective anti-viral treatment regimens. Also, by avoiding the administration of ineffective drugs, considerable time and money is saved.

The methods for measuring replication fitness can be adapted to other viruses, including, but not limited to hepadnaviruses (e.g., human hepatitis B virus), flaviviruses (e.g., human hepatitis C virus) and herpesviruses (e.g., human cytomegalovirus).

This invention further relates to a method for measuring the replication fitness of HIV-1 that exhibits reduced drug susceptibility to reverse transcriptase inhibitors and protease inhibitors. The methods for measuring replication fitness can be adapted to other classes of inhibitors of HIV-1 replication, including, but not limited to, integration, virus assembly, and virus attachment and entry.

The invention further relates to a method for identifying mutations in protease or reverse transcriptase that alter replication fitness.

The methods for identifying mutations that alter replication fitness described herein can be adapted to other components of HIV-1 replication, including, but not limited to, integration, virus assembly, and virus attachment and entry.

The present invention further relates to methods for quantifying the effect that specific mutations in protease or reverse transcriptase have on replication fitness. The methods for quantifying the affect that specific protease and reverse transcriptase mutations have on replication fitness can be adapted to mutations in other viral genes involved in HIV-1 replication, including, but not limited to the gag, pol, and envelope genes.

This invention further relates to the high incidence of patient samples with reduced replication fitness and the general correlation between reduced drug susceptibility and reduced replication fitness. More specifically, the present invention further relates to the occurrence of viruses with reduced fitness in patients receiving protease inhibitor and/or reverse transcriptase inhibitor treatment.

The invention further relates to the incidence of patient samples with reduced replication fitness in which the reduction in fitness is due to altered protease processing of the gag polyprotein (p55).

The invention further relates to the incidence of protease mutations in patient samples that exhibit low, moderate or normal (wild-type) replication fitness.

The invention further relates to protease mutations that are frequently observed, either alone or in combination, in viruses that exhibit reduced replication capacity.

The invention also relates to the incidence of patient samples with reduced replication fitness in which the reduction in fitness is due to altered reverse transcriptase activity.

The invention relates to the occurrence of viruses with reduced replication fitness in patients failing antiretroviral drug treatment.

The invention further relates to a method for using replication fitness measurements to guide the treatment of HIV-1, for example, to methods for using replication fitness measurements to guide the treatment of patients failing antiretroviral drug treatment or for using replication fitness measurements to guide the treatment of patients newly infected with HIV-1. The methods for using replication fitness measurements to guide the treatment of HIV-1 can be adapted to other viruses, including, but not limited to hepadnaviruses (e.g., human hepatitis B virus), flaviviruses (e.g., human hepatitis C virus) and herpesviruses (e.g., human cytomegalovirus).

In one aspect, the present invention provides a method for determining whether a HIV, e.g., HIV-1, has an increased likelihood of having an impaired replication capacity, comprising: detecting whether the reverse transcriptase encoded by said HIV exhibits the presence or absence of a mutation associated with impaired replication capacity at amino acid position 98, 100, 101, 103, 106, 108, 179, 181, 188, 190, 225 or 236 of the amino acid sequence of said reverse transcriptase, wherein the presence of said mutation indicates that the HIV has an increased likelihood of having impaired replication capacity, with the proviso that said mutation is not P236L.

In another aspect, the present invention provides a method for determining whether a HIV, e.g., HIV-1, has an increased likelihood of having an impaired replication capacity, comprising detecting whether the reverse transcriptase encoded by said HIV exhibits the presence or absence of a mutation selected from the group consisting of A98G, L100I, K101E, K103N, V106A, V106I , V106M, Y181C, Y188A, Y188C, Y188, Y188L, G190A, G190C, G190E, G190T, G190V, G190Q, G190S and G190V of the amino acid sequence of said reverse transcriptase, wherein the presence of said mutation indicates that the HIV has an increased likelihood of having impaired replication capacity.

In another aspect, the present invention provides a method for determining whether a subject has an HIV, e.g., HIV-1, with an increased likelihood of having an impaired replication capacity, comprising detecting whether the reverse transcriptase encoded by said HIV exhibits the presence or absence of a mutation associated with impaired replication capacity at amino acid position 98, 100, 101, 103, 106, 108, 179, 181, 188, 190, 225 or 236 of the amino acid sequence of said reverse transcriptase, wherein the presence of said mutation indicates that the HIV has an increased likelihood of having impaired replication capacity, with the proviso that said mutation is not P236L.

In another embodiment, said method comprises detecting the presence or absence of a mutation associated with impaired replication capacity at at least 2, 3, 4, 5, 6, 7, 8, 9, 10, 11 or 12 amino acid positions. In general, the methods can comprise detecting the presence or absence of any combinations of mutations listed herein associated with impaired replication capacity. For example, the method can comprise detecting the presence or absence of a mutation at at least two amino acid positions, such as amino acid positions 106 and 181, 103 and 190, 103 and 236, 181 and 236, 103 and 188, 103 and 181, 100 and 103, or 98 and 181. In certain embodiments, such methods can comprise detecting the presence or absence of V106A and Y181C; K103N and G109S; P236L and K103N; P236L and Y181C; K103N and G190A; K103N and Y181C; K103N and Y188L; L100I and K103N; or Y181C and A98G.

Moreover, the method can comprise detecting the presence or absence of a mutation at at least three amino acid positions, such as amino acid positions 103, 181 and 236; 100, 103, and 190; or 103, 181 and 225. In certain embodiments, such methods can comprise detecting the presence or absence of P236L, K103N and Y181C; L100I, K103N and G190S; or K103N, Y181C and P225H.

In another aspect, the present invention provides a method for determining whether a subject has an HIV, e.g., HIV-1, with an increased likelihood of having an impaired replication capacity, comprising detecting whether the reverse transcriptase encoded by said HIV exhibits the presence or absence of a mutation selected from the group consisting of A98G, L100I, K101E, K103N, V106A, V106I, V106M, Y181C, Y188A, Y188C, Y188H, Y188L, G190A, G190C, G190E, G190T, G190V, G190Q, G190S and G190V of the amino acid sequence of said reverse transcriptase, wherein the presence of said mutation indicates that the HIV has an increased likelihood of having impaired replication capacity.

In one embodiment, said mutation confers resistance to a non-nucleoside reverse transcriptase inhibitor (“NNRTI”). In another embodiment, said human immunodeficiency virus is human immunodeficiency virus type 1 (HIV-1). In another embodiment, said NNRTI is nevirapine (“NVP”), delavirdine (“DLV”) or efavirenz (“EFV”). In another embodiment, said presence or absence of said mutation in said reverse transcriptase (“RT”) is detected by hybridization with a sequence-specific oligonucleotide probe to a nucleic acid sequence of said human immunodeficiency virus encoding said mutation, wherein the occurrence of hybridization indicates said presence or absence of said mutation. In another embodiment, said presence or absence of said mutation in said RT is detected by determining the nucleic acid sequence encoding said mutation. In another embodiment, said presence or absence of said mutation in said RT is detected by amplifying the nucleic acid by, e.g., PCR. In another embodiment, said subject is undergoing or has undergone prior treatment with an anti-viral drug. In one embodiment, the anti-viral drug is an NNRTI.

In another aspect, the present invention provides an isolated oligonucleotide between about 10 and about 40 nucleotides long encoding a portion of a HIV reverse transcriptase in a HIV that comprises a mutation at amino acid position 98, 100, 101, 103, 106, 108, 179, 181, 188, 190, 225 or 236 of an amino acid sequence of said reverse transcriptase in said HIV, wherein the mutation is associated with reduced susceptibility to a protease inhibitor, with the proviso that said mutation is not P236L.

### 4. BRIEF DESCRIPTION OF THE FIGURES

FIG. 1 present a diagrammatic representation of a replication capacity assay.

FIG. 2 presents a chart summarizing the correlation between the presence of mutations and pairs of mutations and replication capacity.

FIG. 3 presents charts summarizing the replication capacity of recombinant viruses containing different amino acid substitutions at the same position within reverse transcriptase.

FIG. 4 presents charts demonstrating that replication capacity measurements made using the replication capacity assay are consistent with measurements made using a replication competition assay.

FIG. 5 presents charts demonstrating that L74V partially restores the impaired replication capacity of recombinant viruses containing G190 mutations.

FIG. 6 presents a chart demonstrating the replication capacity of patient derived viruses that contain detrimental G 190 mutations.

FIG. 7 presents a chart showing that the effects of K103N and other NNRTI mutations are approximately additive.

FIG. 8 presents charts illustrating the relationship between drug concentration and replication capacity for different substitution mutations at the same amino acid position in reverse transcriptase. FIG. 8A illustrates the relationship between delavirdine and replication capacity for different substitution mutations at the same amino acid position in reverse transcriptase. FIG. 8B illustrates the relationship between efavirenz and replication capacity for different substitution mutations at the same amino acid position in reverse transcriptase. FIG. 8C illustrates the relationship between nevirapine and replication capacity for different substitution mutations at the same amino acid position in reverse transcriptase.

FIG. 9 presents charts illustrating the NNRTI susceptibility distribution in the absence (white boxes) and presence (grey boxes) of K101P.

FIG. 10 presents charts illustrating the NNRTI susceptibility distribution in the absence (white boxes) or presence (grey boxes) of K103R+V179D.

### 5. DETAILED DESCRIPTION OF THE INVENTION

The present invention provides methods and compositions for developing and using methods and compositions for determining the replication capacity of a non-nucleoside reverse transcriptase inhibitor (NNRTI) resistant virus. The methods and compositions are based on an analysis of a panel of recombinant virus vectors created using site-directed mutagenesis containing one or more reverse transcriptase (RT) amino acid substitutions. The methods and compositions of the invention significantly improve the quality of life of a patient by providing information to the clinician useful for the design of more effective anti-viral treatment regimens. Also, by avoiding the administration of ineffective drugs, considerable time and money is saved.

5.1 Abbreviations

“NRTI” is an abbreviation for nucleoside reverse transcriptase inhibitor.

“NNRTI” is an abbreviation for non nucleoside reverse transcriptase inhibitor.

“RT” is an abbreviation for reverse transcriptase.

“PCR” is an abbreviation for “polymerase chain reaction.”

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N->C direction, in accordance with common practice.

Individual amino acids in a sequence are represented herein as AN, wherein A is the standard-one letter symbol for the amino acid in the sequence, and N is the position in the sequence. Mutations are represented herein as A₁NA₂, wherein A₁ is the standard one letter symbol for the amino acid in the reference protein sequence, A₂ is the standard one letter symbol for the amino acid in the mutated protein sequence, and N is the position in the amino acid sequence. For example, a G25M mutation represents a change from glycine to methionine at amino acid position 25. Mutations may also be represented herein as NA₂, wherein N is the position in the amino acid sequence and A₂ is the standard one letter symbol for the amino acid in the mutated protein sequence (e.g., 25M, for a change from the wild-type amino acid to methionine at amino acid position 25). Additionally, mutations may also be represented herein as A₁N, wherein A₁ is the standard one letter symbol for the amino acid in the reference protein sequence and N is the position in the amino acid sequence (e.g., G25 represents a change from glycine to any amino acid at amino acid position 25). This notation is typically used when the amino acid in the mutated protein sequence is either not known or, if the amino acid in the mutated protein sequence could be any amino acid, except that found in the reference protein sequence. The amino acid positions are numbered based on the full-length sequence of the protein from which the region encompassing the mutation is derived. Representations of nucleotides and point mutations in DNA sequences are analogous.

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Unless specified otherwise, single-stranded nucleic acid sequences that are represented as a series of one-letter abbreviations, and the top strand of double-stranded sequences, are presented in the 5′->3′ direction.

5.2 Definitions

As used herein, the following terms shall have the following meanings:

Unless otherwise specified, “primary mutation” refers to a mutation that affects the enzyme active site, i.e. at those amino acid positions that are involved in the enzyme-substrate complex, or that reproducibly appears in an early round of replication when a virus is subject to the selective pressure of an anti-viral agent, or, that has a large effect on phenotypic susceptibility to an anti-viral agent.

“Secondary Mutation” refers to a mutation that is not a primary mutation and that contributes to reduced susceptibility or compensates for gross defects imposed by a primary mutation.

A “phenotypic assay” is a test that measures the sensitivity of a virus (such as HIV) to a specific anti-viral agent.

A “genotypic assay” is a test that determines a genetic sequence of an organism, a part of an organism, a gene or a part of a gene. Such assays are frequently performed in HIV to establish whether certain mutations are associated with drug resistance are present.

As used herein, “genotypic data” are data about the genotype of, for example, a virus. Examples of genotypic data include, but are not limited to, the nucleotide or amino acid sequence of a virus, a part of a virus, a viral gene, a part of a viral gene, or the identity of one or more nucleotides or amino acid residues in a viral nucleic acid or protein.

“Susceptibility” refers to a virus' response to a particular drug. A virus that has decreased or reduced susceptibility to a drug has an increased resistance or decreased sensitivity to the drug. A virus that has increased or enhanced or greater susceptibility to a drug has an increased sensitivity or decreased resistance to the drug.

Phenotypic susceptibility of a virus to a given drug is a continuum. Nonetheless, it is practically useful to define a threshold or thresholds to simplify interpretation of a particular fold-change result. For drugs where sufficient clinical outcome data have been gathered, it is possible to define a “clinical cutoff value,” as below.

“Clinical Cutoff Value” refers to a specific point at which resistance begins and sensitivity ends. It is defined by the drug susceptibility level at which a patient's probability of treatment failure with a particular drug significantly increases. The cutoff value is different for different anti-viral agents, as determined in clinical studies. Clinical cutoff values are determined in clinical trials by evaluating resistance and outcomes data. Drug susceptibility (phenotypic) is measured at treatment initiation. Treatment response, such as change in viral load, is monitored at predetermined time points through the course of the treatment. The drug susceptibility is correlated with treatment response and the clinical cutoff value is determined by resistance levels associated with treatment failure (statistical analysis of overall trial results).

“IC_{n”}, refers to Inhibitory Concentration. It is the concentration of drug in the patient's blood or in vitro needed to suppress the reproduction of a disease-causing microorganism (such as HIV) by n %. Thus, “IC_{50”} refers to the concentration of an anti-viral agent at which virus replication is inhibited by 50% of the level observed in the absence of the drug. “Patient IC_{50”} refers to the drug concentration required to inhibit replication of the virus from a patient by 50% and “reference IC_{50”}, refers to the drug concentration required to inhibit replication of a reference or wild-type virus by 50%. Similarly, “IC_{90 ”} refers to the concentration of an anti-viral agent at which 90% of virus replication is inhibited.

A “fold change” is a numeric comparison of the drug susceptibility of a patient virus and a drug-sensitive reference virus. It is the ratio of the Patient IC₅₀ to the drug-sensitive reference IC₅₀, i.e., Patient IC₅₀/Reference IC₅₀=Fold Change (“FC”). A fold change of 1.0 indicates that the patient virus exhibits the same degree of drug susceptibility as the drug-sensitive reference virus. A fold change less than 1 indicates the patient virus is more sensitive than the drug-sensitive reference virus. A fold change greater than 1 indicates the patient virus is less susceptible than the drug-sensitive reference virus. A fold change equal to or greater than the clinical cutoff value means the patient virus has a lower probability of response to that drug. A fold change less than the clinical cutoff value means the patient virus is sensitive to that drug.

A virus has an “increased likelihood of having impaired replication capacity” if the virus has a property, for example, a mutation, that is correlated with an impaired replication capacity. A property of a virus is correlated with an impaired replication capacity if a population of viruses having the property has, on average, an impaired replication capacity relative to that of an otherwise similar population of viruses lacking the property. Thus, the correlation between the presence of the property and impaired replication capacity need not be absolute, nor is there a requirement that the property is necessary (i.e., that the property plays a causal role in impairing replication capacity) or sufficient (i.e., that the presence of the property alone is sufficient) for impairing replication capacity.

The term “% sequence homology” is used interchangeably herein with the terms “% homology,” “% sequence identity” and “% identity” and refers to the level of amino acid sequence identity between two or more peptide sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 60, 70, 80, 85, 90, 95, 98% or more sequence identity to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at http://www ncbi.nlm.nih.gov/BLAST/. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Altschul, et al., 1997.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with hydronium ion. Genetically encoded basic amino acids include Arg (R), His (H) and Lys (K).

A “mutation” is a change in an amino acid sequence or in a corresponding nucleic acid sequence relative to a reference nucleic acid or polypeptide. For embodiments of the invention comprising HIV protease or reverse transcriptase, the reference nucleic acid encoding protease or reverse transcriptase is the protease or reverse transcriptase coding sequence, respectively, present in NL4-3 HIV (GenBank Accession No. AF324493). Likewise, the reference protease or reverse transcriptase polypeptide is that encoded by the NL4-3 HIV sequence. Although the amino acid sequence of a peptide can be determined directly by, for example, Edman degradation or mass spectroscopy, more typically, the amino sequence of a peptide is inferred from the nucleotide sequence of a nucleic acid that encodes the peptide. Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY).

A “resistance-associated mutation” (“RAM”) in a virus is a mutation correlated with reduced susceptibility of the virus to anti-viral agents. A RAM can be found in several viruses, including, but not limited to a human immunodeficiency virus (“HIV”). Such mutations can be found in one or more of the viral proteins, for example, in the protease, integrase, envelope or reverse transcriptase of HIV. A RAM is defined relative to a reference strain. For embodiments of the invention comprising HIV protease, the reference protease is the protease present in NL4-3 HIV (GenBank Accession No. AF324493).

A “mutant” is a virus, gene or protein having a sequence that has one or more changes relative to a reference virus, gene or protein.

The terms “peptide,” “polypeptide” and “protein” are used interchangeably throughout.

The terms “reference” and “wild-type” are used interchangeably throughout.

The terms “polynucleotide,” “oligonucleotide” and “nucleic acid” are used interchangeably throughout.

5.3 Mutations Associated with Impaired Replication Capacity

The present invention provides nucleic acids and polypeptides comprising a mutation in the reverse transcriptase of HIV associated with resistance to a NNRTI and with impaired replication capacity. Preferably, the HIV is human immunodeficiency virus type 1 (“HIV-1”). Examples of NNRTI include, but are not limited to, nevirapine (“NVP”), delavirdine (“DLV”) and efavirenz (“EFV”).

In one aspect, the present invention provides peptides, polypeptides or proteins comprising a mutation in the reverse transcriptase of HIV associated with resistance to a NNRTI and with impaired replication capacity. Polypeptides of the invention include peptides, polypeptides and proteins that are modified or derived from these polypeptides. In one embodiment, the polypeptide comprises post-translational modifications.

In one embodiment, the invention provides a polypeptide derived from the HIV reverse transcriptase and comprising a mutation at an amino acid position selected from the group consisting of 98, 100, 101, 103, 106, 108, 179, 181, 188, 190, 225 and 236. In a more particularly defined embodiment, the mutation is selected from the group of mutations consisting of A98G, L100I, K101E, K103N, V106A, V106I, V106M, Y181C, Y188A, Y188C, Y188H, Y188L, G190A, G190C, G190E, G190T, G190V, G190Q, G190S and G190V.

In another embodiment, the invention provides a polypeptide derived from the HIV reverse transcriptase and comprising a combination of mutations at two or more amino acid positions. Examples of such combinations include, but are not limited to, P236L, K103N and Y181C; V106A and Y181C; K103N and G190S; P236L and K103N; L100I, K103N and G190S; P236L and Y181C; K103N and G190A; K103N and Y188L; K103N and Y181C; L100I and K103N; K103N, Y181C and 225H; Y181C and A98G.

In another aspect, the present invention provides polynucleotides, oligonucleotides or nucleic acids encoding or relating to a polypeptide of the invention or a biologically active portion thereof, including, for example, nucleic acid molecules sufficient for use as hybridization probes, PCR primers or sequencing primers for identifying, analyzing, mutating or amplifying the nucleic acids of the invention.

The nucleic acid can be any length. The nucleic acid can be, for example, at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 100, 110, 120, 125, 150, 175, 200, 250, 300, 350, 375, 400, 425, 450, 475 or 500 nucleotides in length. The nucleic acid can be, for example, less than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 100, 110, 120, 125, 150, 175, 200, 250, 300, 350, 375, 400, 425, 450, 475, 500, 525, 550, 575, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500 or 10000 nucleotides in length. In a preferred embodiment, the nucleic acid has a length and a sequence suitable for detecting a mutation described herein, for example, as a probe or a primer.

In another embodiment, the present invention provides nucleic acid molecules that are suitable for use as primers or hybridization probes for the detection of nucleic acid sequences of the invention. A nucleic acid molecule of the invention can comprise only a portion of a nucleic acid sequence encoding a full length polypeptide of the invention for example, a fragment that can be used as a probe or primer or a fragment encoding a biologically active portion of a polypeptide of the invention. The probe can comprise a labeled group attached thereto, e.g., a radioisotope, a fluorescent compound, an enzyme, or an enzyme co-factor. In various embodiments, the nucleic acid molecules of the invention can be modified at the base moiety, sugar moiety or phosphate backbone.

5.4 Finding Mutations Associated with Impaired Replication Capacity

In another aspect, the present invention provides methods for finding mutations associated with impaired replication capacity in a virus or a derivative of the virus.

5.4.1 The Virus and Viral Samples

An impaired replication capacity-associated mutation according to the present invention can be present in any type of virus, for example, any virus found in animals. In one embodiment of the invention, the virus includes viruses known to infect mammals, including dogs, cats, horses, sheep, cows etc. In a preferred embodiment, the virus is known to infect primates. In an even more preferred embodiment the virus is known to infect humans. Examples of human viruses include, but are not limited to, human immunodeficiency virus (“HIV”), herpes simplex virus, cytomegalovirus virus, varicella zoster virus, other human herpes viruses, influenza A virus, respiratory syncytial virus, hepatitis A, B, and C viruses, rhinovirus, and human papilloma virus. In a preferred embodiment of the invention, the virus is HIV. Preferably, the virus is human immunodeficiency virus type 1 (“HIV-1”). The foregoing are representative of certain viruses for which there is presently available anti-viral chemotherapy and represent the viral families retroviridae, herpesviridae, orthomyxoviridae, paramxyxovirus, picornavirus, flavivirus, pneumovirus and hepadnaviridae. This invention can be used with other viral infections due to other viruses within these families as well as viral infections arising from viruses in other viral families for which there is or there is not a currently available therapy.

An impaired replication capacity-associated mutation according to the present invention can be found in a viral sample obtained by any means known in the art for obtaining viral samples. Such methods include, but are not limited to, obtaining a viral sample from a human or an animal infected with the virus or obtaining a viral sample from a viral culture. In one embodiment, the viral sample is obtained from a human individual infected with the virus. The viral sample could be obtained from any part of the infected individual's body or any secretion expected to contain the virus. Examples of such parts include, but are not limited to blood, serum, plasma, sputum, lymphatic fluid, semen, vaginal mucus and samples of other bodily fluids. In a preferred embodiment, the sample is a blood, serum or plasma sample.

In another embodiment, an impaired replication capacity-associated mutation according to the present invention is present in a virus that can be obtained from a culture. In some embodiments, the culture can be obtained from a laboratory. In other embodiments, the culture can be obtained from a collection, for example, the American Type Culture Collection.

In certain embodiments, an impaired replication capacity-associated mutation according to the present invention is present in a derivative of a virus. In one embodiment, the derivative of the virus is not itself pathogenic. In another embodiment, the derivative of the virus is a plasmid-based system, wherein replication of the plasmid or of a cell transfected with the plasmid is affected by the presence or absence of the selective pressure, such that mutations are selected that increase resistance to the selective pressure. In some embodiments, the derivative of the virus comprises the nucleic acids or proteins of interest, for example, those nucleic acids or proteins to be targeted by an anti-viral treatment. In one embodiment, the genes of interest can be incorporated into a vector. See, e.g., U.S. Pat. Nos. 5,837,464 and 6,242,187 and PCT publication, WO 99/67427, each of which is incorporated herein by reference. In a preferred embodiment, the genes can be those that encode for a protease or reverse transcriptase.

In another embodiment, the intact virus need not be used. Instead, a part of the virus incorporated into a vector can be used. Preferably that part of the virus is used that is targeted by an anti-viral drug.

In another embodiment, an impaired replication capacity-associated mutation according to the present invention is present in a genetically modified virus. The virus can be genetically modified using any method known in the art for genetically modifying a virus. For example, the virus can be grown for a desired number of generations in a laboratory culture. In one embodiment, no selective pressure is applied (i.e., the virus is not subjected to a treatment that favors the replication of viruses with certain characteristics), and new mutations accumulate through random genetic drift. In another embodiment, a selective pressure is applied to the virus as it is grown in culture (i.e., the virus is grown under conditions that favor the replication of viruses having one or more characteristics). In one embodiment, the selective pressure is an anti-viral treatment. Any known anti-viral treatment can be used as the selective pressure. In one embodiment, the virus is HIV and the selective pressure is a NNRTI. In another embodiment, the virus is HIV-1 and the selective pressure is a NNRTI. Any NNRTI can be used to apply the selective pressure. Examples of NNRTIs include, but are not limited to, NVP, DLV and EFV. By treating HIV cultured in vitro with a NNRTI, one can select for mutant strains of HIV that have an increased resistance to the NNRTI. The stringency of the selective pressure can be manipulated to increase or decrease the survival of viruses not having the selected-for characteristic.

In another aspect, an impaired replication capacity-associated mutation according to the present invention is made by mutagenizing a virus, a viral genome, or a part of a viral genome. Any method of mutagenesis known in the art can be used for this purpose. In one embodiment, the mutagenesis is essentially random. In another embodiment, the essentially random mutagenesis is performed by exposing the virus, viral genome or part of the viral genome to a mutagenic treatment. In another embodiment, a gene that encodes a viral protein that is the target of an anti-viral therapy is mutagenized. Examples of essentially random mutagenic treatments include, for example, exposure to mutagenic substances (e.g., ethidium bromide, ethylmethanesulphonate, ethyl nitroso urea (ENU) etc.) radiation (e.g., ultraviolet light), the insertion and/or removal of transposable elements (e.g., Tn5, Tn10), or replication in a cell, cell extract, or in vitro replication system that has an increased rate of mutagenesis. See, e.g., Russell et al., 1979, Proc. Nat. Acad. Sci. USA 76:5918-5922; Russell, W., 1982, Environmental Mutagens and Carcinogens: Proceedings of the Third International Conference on Environmental Mutagens. One of skill in the art will appreciate that while each of these methods of mutagenesis is essentially random, at a molecular level, each has its own preferred targets.

In another aspect, an impaired replication capacity-associated mutation is made using site-directed mutagenesis. Any method of site-directed mutagenesis known in the art can be used (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY). The site directed mutagenesis can be directed to, e.g., a particular gene or genomic region, a particular part of a gene or genomic region, or one or a few particular nucleotides within a gene or genomic region. In one embodiment, the site directed mutagenesis is directed to a viral genomic region, gene, gene fragment, or nucleotide based on one or more criteria. In one embodiment, a gene or a portion of a gene is subjected to site-directed mutagenesis because it encodes a protein that is known or suspected to be a target of an anti-viral therapy, e.g., the gene encoding the HIV reverse transcriptase. In another embodiment, a portion of a gene, or one or a few nucleotides within a gene, are selected for site-directed mutagenesis. In one embodiment, the nucleotides to be mutagenized encode amino acid residues that are known or suspected to interact with an anti-viral compound. In another embodiment, the nucleotides to be mutagenized encode amino acid residues that are known or suspected to be mutated in viral strains having an impaired replication capacity. In another embodiment, the mutagenized nucleotides encode amino acid residues that are adjacent to or near in the primary sequence of the protein residues known or suspected to interact with an anti-viral compound or known or suspected to be mutated in viral strains having an impaired replication capacity. In another embodiment, the mutagenized nucleotides encode amino acid residues that are adjacent to or near to in the secondary, tertiary or quaternary structure of the protein residues known or suspected to interact with an anti-viral compound or known or suspected to be mutated in viral strains having an impaired replication capacity. In another embodiment, the mutagenized nucleotides encode amino acid residues in or near the active site of a protein that is known or suspected to bind to an anti-viral compound. See e.g., Sarkar and Sommer, 1990, Biotechniques, 8:404-407.

5.4.2 Detecting the Presence or Absence of Mutations in a Virus

The presence or absence of an impaired replication capacity-associated mutation according to the present invention in a virus can be detected by any means known in the art for detecting a mutation. The mutation can be detected in the viral gene that encodes a particular protein, or in the protein itself, i.e., in the amino acid sequence of the protein.

In one embodiment, the mutation is in the viral genome. Such a mutation can be in, for example, a gene encoding a viral protein, in a cis or trans acting regulatory sequence of a gene encoding a viral protein, an intergenic sequence, or an intron sequence. The mutation can affect any aspect of the structure, function, replication or environment of the virus that changes its susceptibility to an anti-viral treatment. In one embodiment, the mutation is in a gene encoding a viral protein that is the target of an anti-viral treatment.

A mutation within a viral gene can be detected by utilizing a number of techniques. Viral DNA or RNA can be used as the starting point for such assay techniques, and may be isolated according to standard procedures which are well known to those of skill in the art.

The detection of a mutation in specific nucleic acid sequences, such as in a particular region of a viral gene, can be accomplished by a variety of methods including, but not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. U.S.A. 80:1579-83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S. 92:87-91) cleavage of heteroduplex DNA, methods based on oligonucleotide-specific primer extension (Syvänen et al., 1990, Genomics 8:684-692), genetic bit analysis (Nikiforov et al., 1994, Nucl Acids. Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids. Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J. Mol. Biol. 98:503-517), single stranded conformational polymorphism analyses (SSCP) (Orita et al., 1989, Proc. Natl. Acad. Sci. USA 86:2766-2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods for the detection of a gene-specific mutation can involve for example, contacting and incubating the viral nucleic acids with one or more labeled nucleic acid reagents including recombinant DNA molecules, cloned genes or degenerate variants thereof, under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microtiter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, the nucleic acid can be sequenced by any sequencing method known in the art. For example, the viral DNA can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also the techniques described in Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY.

Antibodies directed against the viral gene products, i.e., viral proteins or viral peptide fragments can also be used to detect mutations in the viral proteins. Alternatively, the viral protein or peptide fragments of interest can be sequenced by any sequencing method known in the art in order to yield the amino acid sequence of the protein of interest. An example of such a method is the Edman degradation method which can be used to sequence small proteins or polypeptides. Larger proteins can be initially cleaved by chemical or enzymatic reagents known in the art, for example, cyanogen bromide, hydroxylamine, trypsin or chymotrypsin, and then sequenced by the Edman degradation method.

5.5 Measuring Phenotypic Susceptibility of a Mutant Virus

Any method known in the art can be used to determine the phenotypic susceptibility of a mutant virus or population of viruses to an anti-viral therapy. See e.g., U.S. Pat. Nos. 5,837,464 and 6,242,187, incorporated herein by reference in their entirities. In some embodiments a phenotypic analysis is performed, i.e., the susceptibility of the virus to a given anti-viral agent is assayed with respect to the susceptibility of a reference virus without the mutations. This is a direct, quantitative measure of drug susceptibility and can be performed by any method known in the art to determine the susceptibility of a virus to an anti-viral agent. An example of such methods includes, but is not limited to, determining the fold change in IC₅₀ values with respect to a reference virus. Phenotypic testing measures the ability of a specific viral strain to grow in vitro in the presence of a drug inhibitor. A virus is less susceptible to a particular drug when more of the drug is required to inhibit viral activity, versus the amount of drug required to inhibit the reference virus.

In one embodiment, a phenotypic analysis is performed and used to calculate the IC₅₀ or IC₉₀ of a drug for a viral strain. The results of the analysis can also be presented as fold-change in IC₅₀ or IC₉₀ for each viral strain as compared with a drug-susceptible control strain or a prior viral strain from the same patient. Because the virus is directly exposed to each of the available anti-viral medications, results can be directly linked to treatment response. For example, if the patient virus shows resistance to a particular drug, that drug is avoided or omitted from the patient's treatment regimen, allowing the physician to design a treatment plan that is more likely to be effective for a longer period of time.

In another embodiment, the phenotypic analysis is performed using recombinant virus assays (“RVAs”). RVAs use virus stocks generated by homologous recombination between viral vectors and viral gene sequences, amplified from the patient virus. In some embodiments, the viral vector is a HIV vector and the viral gene sequences are protease and/or reverse transcriptase sequences.

In a preferred embodiment, the phenotypic analysis is performed using PHENOSENSE™ (ViroLogic Inc., South San Francisco, Calif.). See Petropoulos et al., 2000, Antimicrob. Agents Chemother. 44:920-928; U.S. Pat. Nos. 5,837,464 and 6,242,187. PHENOSENSE™ is a phenotypic assay that achieves the benefits of phenotypic testing and overcomes the drawbacks of previous assays. Because the assay has been automated, PHENOSENSE™ offers higher throughput under controlled conditions. The result is an assay that accurately defines the susceptibility profile of a patient's HIV isolates to all currently available antiretroviral drugs, and delivers results directly to the physician within about 10 to about 15 days of sample receipt. PHENOSENSE™ is accurate and can obtain results with only one round of viral replication, thereby avoiding selection of subpopulations of virus. The results are quantitative, measuring varying degrees of drug susceptibility, and sensitive—the test can be performed on blood specimens with a viral load of about 500 copies/mL and can detect minority populations of some drug-resistant virus at concentrations of 10% or less of total viral population. Furthermore, the results are reproducible and can vary by less than about 1.4-2.5 fold, depending on the drug, in about 95% of the assays performed.

PHENOSENSE™ can be used with nucleic acids from amplified viral gene sequences. As discussed in Section 5.4.1, the sample containing the virus may be a sample from a human or an animal infected with the virus or a sample from a culture of viral cells. In one embodiment, the viral sample comprises a genetically modified laboratory strain.

A resistance test vector (“RTV”) can then be constructed by incorporating the amplified viral gene sequences into a replication defective viral vector by using any method known in the art of incorporating gene sequences into a vector. In one embodiment, restrictions enzymes and conventional cloning methods are used. See Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY. In a preferred embodiment, ApaI and PinAI restriction enzymes are used. Preferably, the replication defective viral vector is the indicator gene viral vector (“IGVV”). In a preferred embodiment, the viral vector contains a means for detecting replication of the RTV. Preferably, the viral vector contains a luciferase expression cassette.

The assay can be performed by first co-transfecting host cells with RTV DNA and a plasmid that expresses the envelope proteins of another retrovirus, for example, amphotropic murine leukemia virus (MLV). Following transfection, virus particles can be harvested and used to infect fresh target cells. The completion of a single round of viral replication can be detected by the means for detecting replication contained in the vector. In a preferred embodiment, the completion of a single round of viral replication results in the production of luciferase. Serial concentrations of anti-viral agents can be added at either the transfection step or the infection step.

Susceptibility to the anti-viral agent can be measured by comparing the replication of the vector in the presence and absence of the anti-viral agent. For example, susceptibility to the anti-viral agent can be measured by comparing the luciferase activity in the presence and absence of the anti-viral agent. Susceptible viruses would produce low levels of luciferase activity in the presence of antiviral agents, whereas viruses with reduced susceptibility would produce higher levels of luciferase activity.

In preferred embodiments, PHENOSENSE™ is used in evaluating the phenotypic susceptibility of HIV-1 to anti-viral drugs. Preferably, the anti-viral drug is a NNRTI. In preferred embodiments, the reference viral strain is HIV strain NL4-3 or HXB-2.

In one embodiment, viral nucleic acid, for example, HIV-1 RNA is extracted from plasma samples, and a fragment of, or entire viral genes could be amplified by methods such as, but not limited to PCR. See, e.g., Hertogs et al., 1998, Antimicrob Agents Chemother 42(2):269-76. In one example, a 2.2-kb fragment containing the entire HIV-1 PR- and RT-coding sequence is amplified by nested reverse transcription-PCR. The pool of amplified nucleic acid, for example, the PR-RT-coding sequences, is then cotransfected into a host cell such as CD4+T lymphocytes (MT4) with the pGEMT3deltaPRT plasmid from which most of the PR (codons 10 to 99) and RT (codons 1 to 482) sequences are deleted. Homologous recombination leads to the generation of chimeric viruses containing viral coding sequences, such as the PR- and RT-coding sequences derived from HIV-RNA in plasma. The susceptibilities of the chimeric viruses to all currently available anti-viral agents targeting the products of the transfected genes (proRT and/or PR inhibitors, for example), can be determined by any cell viability assay known in the art. For example, an MT4 cell-3-(4,5-dimeihylthiazol-2-yl)-2,5-diphenyltetrazolium bromide-based cell viability assay can be used in an automated system that allows high sample throughput. The profile of resistance to all the anti-viral agents, such as the RT and PR inhibitors can be displayed graphically in a single PR-RT-Antivirogram.

Other assays for evaluating the phenotypic susceptibility of a virus to anti-viral drugs known to one of skill in the art can be used. See, e.g., Shi and Mellors, 1997, Antimicrob Agents Chemother. 41(12):2781-85; Gervaix et al., 1997, Proc Natl Acad Sci U.S.A. 94(9):4653-8; Race et al., 1999, AIDS 13:2061-2068, incorporated herein by reference in their entireties.

In another embodiment, the susceptibility of a virus to treatment with an anti-viral treatment is determined by assaying the activity of the target of the anti-viral treatment in the presence of the anti-viral treatment. In one embodiment, the virus is HIV, the anti-viral treatment is a protease inhibitor, and the target of the anti-viral treatment is the HIV protease. See, e.g., U.S. Pat. Nos. 5,436,131, 6,103,462, incorporated herein by reference in their entireties.

5.6 Correlating Mutations with Impaired Replication Capacity

Any method known in the art can be used to determine whether a mutation is correlated with an impaired replication capacity. In one embodiment, P values are used to determine the statistical significance of the correlation, such that the smaller the P value, the more significant the measurement. Preferably the P values will be less than 0.05. More preferably, P values will be less than 0.01. P values can be calculated by any means known to one of skill in the art. In one embodiment, P values are calculated using Fisher's Exact Test. See, e.g., David Freedman, Robert Pisani & Roger Purves, 1980, STATISTICS, W. W. Norton, New York.

5.7 Detecting Impaired Replication Capacity

In another aspect, the present invention provides a method for detecting impaired replication capacity in a virus. Impaired replication capacity-associated mutations can be identified using any suitable method, as described above. The presence of an impaired replication capacity-associated mutation in a virus can be detected by any means known in the art, e.g., as discussed above. The presence of an impaired replication capacity-associated mutation in the virus indicates that the virus has an increased likelihood of having an impaired replication capacity. In one embodiment, the virus is human immunodeficiency virus (HIV). In another embodiment, the virus is human immunodeficiency virus type-1 (HIV-1). In another embodiment, the anti-viral treatment is a NNRTI.

In another embodiment, the invention provides a method for determining whether a HIV has an increased likelihood of having an impaired replication capacity, comprising detecting in the RT of said HIV the presence or absence of a mutation associated with impaired replication capacity at amino acid position 98, 100, 101, 103, 106, 108, 179, 181, 188, 190, 225 or 236 of an amino acid sequence of said RT, wherein the presence of said mutation indicates that the HIV has an increased likelihood of having an impaired replication capacity. In another embodiment, the mutation is not P236L.

In another embodiment, the invention provides a method for determining whether a HIV has an increased likelihood of having an impaired replication capacity, comprising detecting in the RT of said HIV the presence or absence of a mutation associated with impaired replication capacity selected from the group consisting of A98G, L100I, K101E, K103N, V106A, V106I, V106M, Y181C, Y188A, Y188C, Y188H, Y188L, G190A, G190C, G190E, G190T, G190V, G190Q, G190S and G190V, wherein the presence of said mutation indicates that the HIV has an increased likelihood of having an impaired replication capacity.

In another embodiment, the invention provides a method for determining whether a HIV from a subject has an increased likelihood of having an impaired replication capacity, comprising detecting in the RT of said HIV the presence or absence of a mutation associated with impaired replication capacity at amino acid position 98, 100, 101, 103, 106, 108, 179, 181, 188, 190, 225 or 236 of an amino acid sequence of said RT, wherein the presence of said mutation indicates that the HIV has an increased likelihood of having an impaired replication capacity. In another embodiment, the mutation is not P236L.

In another embodiment, the invention provides a method for determining whether a HIV from a subject has an increased likelihood of having an impaired replication capacity, comprising detecting in the RT of said HIV the presence or absence of a mutation associated with impaired replication capacity selected from the group consisting of A98G, L100I, K101E, K103N, V106A, V106I, V106M, Y181C, Y188A, Y188C, Y188H, Y188L, G190A, G190C, G190E, G190T, G190V, G190Q, G190S and G190V, wherein the presence of said mutation indicates that the HIV has an increased likelihood of having an impaired replication capacity.

### 6. EXAMPLES

6.1 Example 1

### Measuring Replication Fitness Using Resistance Test Vectors

This example provides methods and compositions for accurately and reproducibly measuring the replication fitness of HIV-1. The methods for measuring replication fitness can be adapted to other viruses, including, but not limited to hepadnaviruses (e.g., human hepatitis B virus), flaviviruses (e.g., human hepatitis C virus) and herpesviruses (e.g., human cytomegalovirus). This example further provides a method for measuring the replication fitness of HIV-1 that exhibits reduced drug susceptibility to reverse transcriptase inhibitors and protease inhibitors. The methods for measuring replication fitness can be adapted to other classes of inhibitors of HIV-1 replication, including, but not limited to integration, virus assembly, and virus attachment and entry.

Replication fitness tests are carried out using the methods for phenotypic drug susceptibility and resistance tests described in U.S. Pat. No. 5,837,464 (International Publication Number WO 97/27319) which is hereby incorporated by reference in its entirety.

Patient-derived segment(s) corresponding to the HIV protease and reverse transcriptase coding regions were either patient-derived segments amplified by the reverse transcription-polymerase chain reaction method (RT-PCR) using viral RNA isolated from viral particles present in the serum of HIV-infected individuals or were mutants of wild type HIV-1 made by site directed mutagenesis of a parental clone of resistance test vector DNA. Resistance test vectors are also referred to as “fitness test vectors” when used to evaluate replication fitness. Isolation of viral RNA was performed using standard procedures (e.g. RNAGENTS™ Total RNA Isolation System, Promega, Madison Wis. or RNAzol, Tel-Test, Friendswood, Tex.). The RT-PCR protocol was divided into two steps. A retroviral reverse transcriptase (e.g. Moloney MuLV reverse transcriptase (Roche Molecular Systems, Inc., Branchburg, N.J.), or avian myeloblastosis virus (AMV) reverse transcriptase, (Boehringer Mannheim, Indianapolis, Ind.)) was used to copy viral RNA into cDNA. The cDNA was then amplified using a thermostable DNA polymerase (e.g. Taq (Roche Molecular Systems, Inc., Branchburg, N.J.), Tth (Roche Molecular Systems, inc., Branchburg, N.J.), PRIMEZYME™ (isolated from Thermus brockianus, Biometra, Gottingen, Germany)) or a combination of thermostable polymerases as described for the performance of “long PCR” (Barnes, W. M., 1994, Proc. Natl. Acad. Sci, USA 91, 2216-20) (e.g. Expand High Fidelity PCR System (Taq+Pwo), (Boehringer Mannheim. Indianapolis, Ind.) OR GENEAMP XL™ PCR kit (Tth+Vent), (Roche Molecular Systems, Inc., Branchburg, N.J.)).

PCR primers were designed to introduce ApaI and AgeI recognition sites into both ends of the PCR product, respectively.

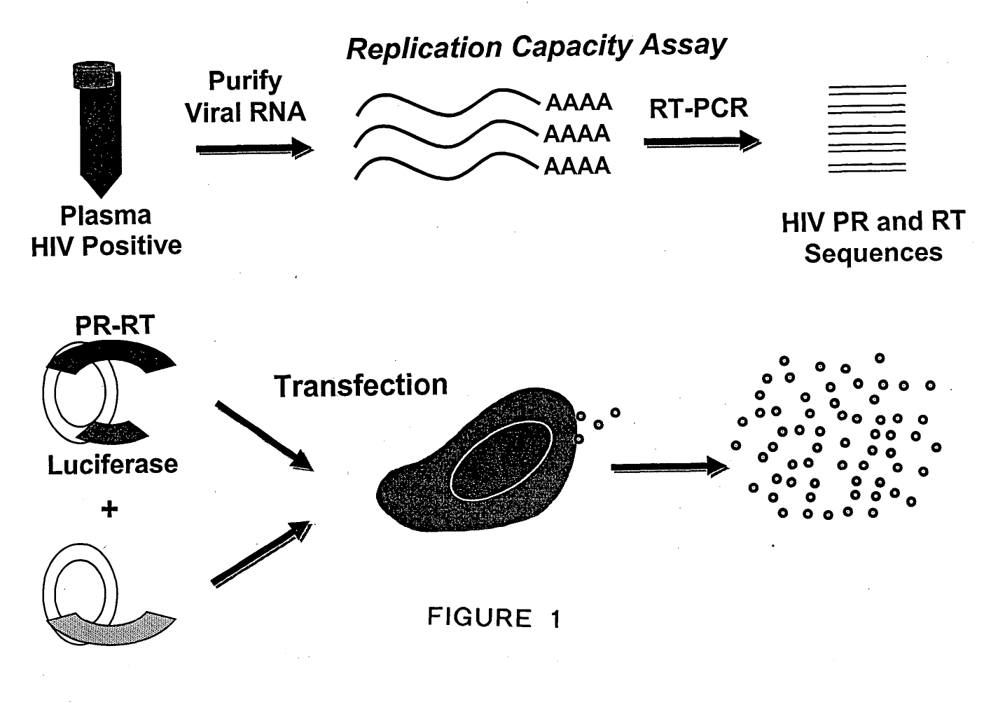

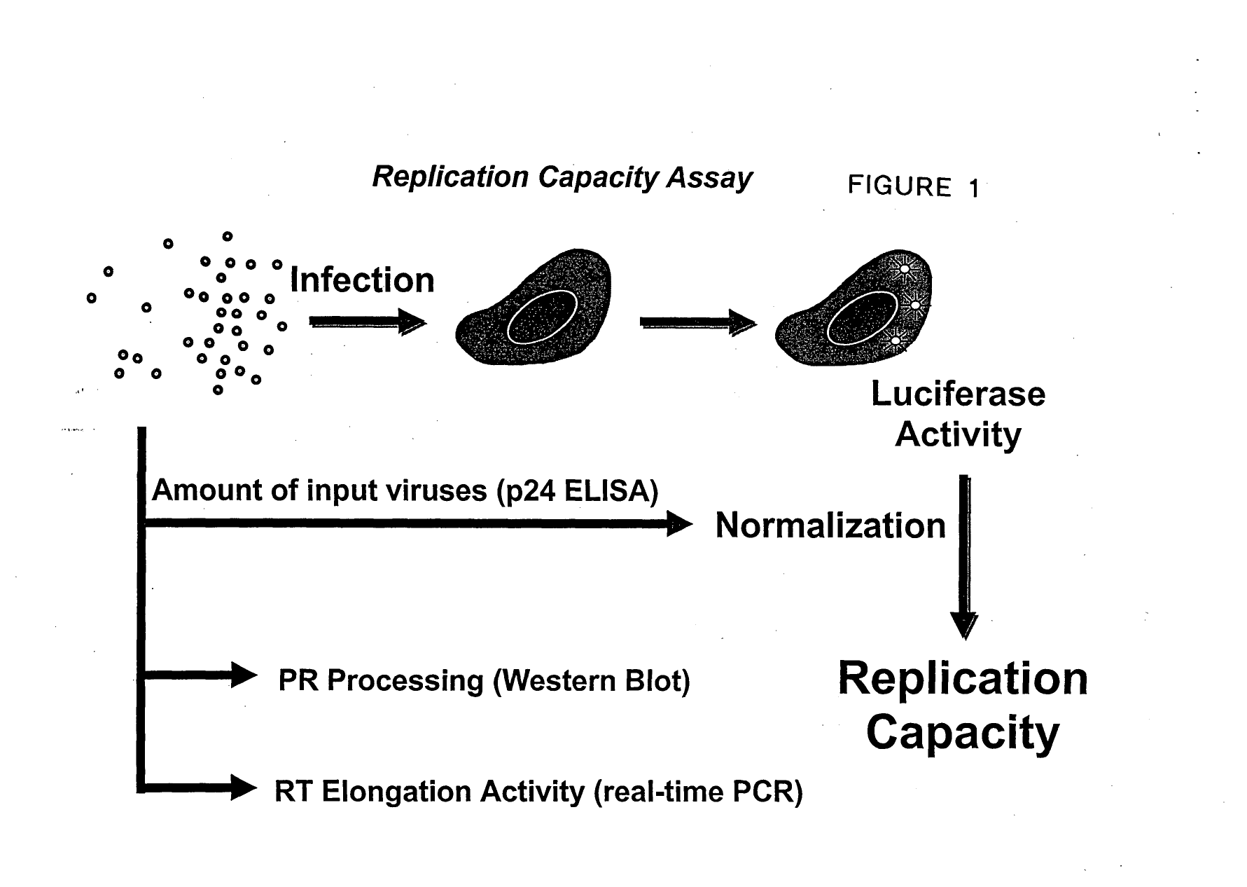

Fitness test vectors incorporating the “test” patient-derived segments were constructed as described in U.S. Pat. No. 5,837,464 (see FIG. 1) using an amplified DNA product of 1.5 kB prepared by RT-PCR using viral RNA as a template and oligonucleotides PCR6 (#1), PDSApa (#2) and PDSAge (#3) as primers, followed by digestion with ApaI and AgeI or the isoschizomer PinA1. To ensure that the plasmid DNA corresponding to the resultant fitness test vector comprises a representative sample of the HIV viral quasi-species present in the serum of a given patient, many (>100) independent E. Coli transformants obtained in the construction of a given fitness test vector were pooled and used for the preparation of plasmid DNA.

A packaging expression vector encoding an amphotrophic MuLV 4070A env gene product enables production in a fitness test vector host cell of fitness test vector viral particles which can efficiently infect human target cells. Fitness test vectors encoding all HIV genes with the exception of env were used to transfect a packaging host cell (once transfected the host cell is referred to as a fitness test vector host cell). The packaging expression vector which encodes the amphotrophic MuLV 4070A env gene product is used with the resistance test vector to enable production in the fitness test vector host cell of infectious pseudotyped fitness test vector viral particles.

Fitness tests performed with fitness test vectors were carried out using packaging host and target host cells consisting of the human embryonic kidney cell line 293.

Fitness tests were carried out with fitness test vectors using two host cell types. Fitness test vector viral particles were produced by a first host cell (the fitness test vector host cell) that was prepared by transfecting a packaging host cell with the fitness test vector and the packaging expression vector. The fitness test vector viral particles were then used to infect a second host cell (the target host cell) in which the expression of the indicator gene is measured (see FIG. 1).

The fitness test vectors containing a functional luciferase gene cassette were constructed and host cells were transfected with the fitness test vector DNA. The fitness test vectors contained patient-derived reverse transcriptase and protease DNA sequences that encode proteins which were either susceptible or resistant to the antiretroviral agents, such as nucleoside reverse transcriptase inhibitors, non-nucleoside reverse transcriptase inhibitors and protease inhibitors.

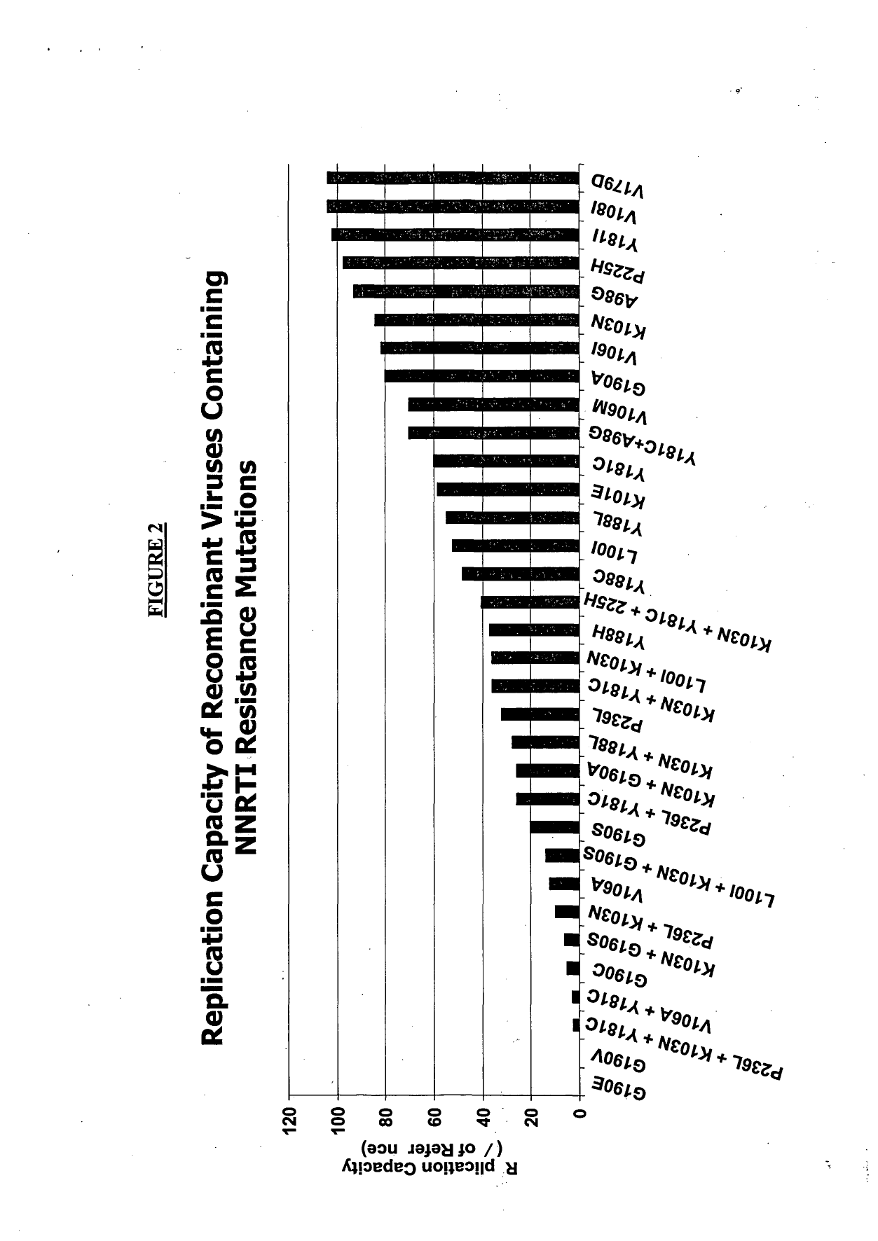

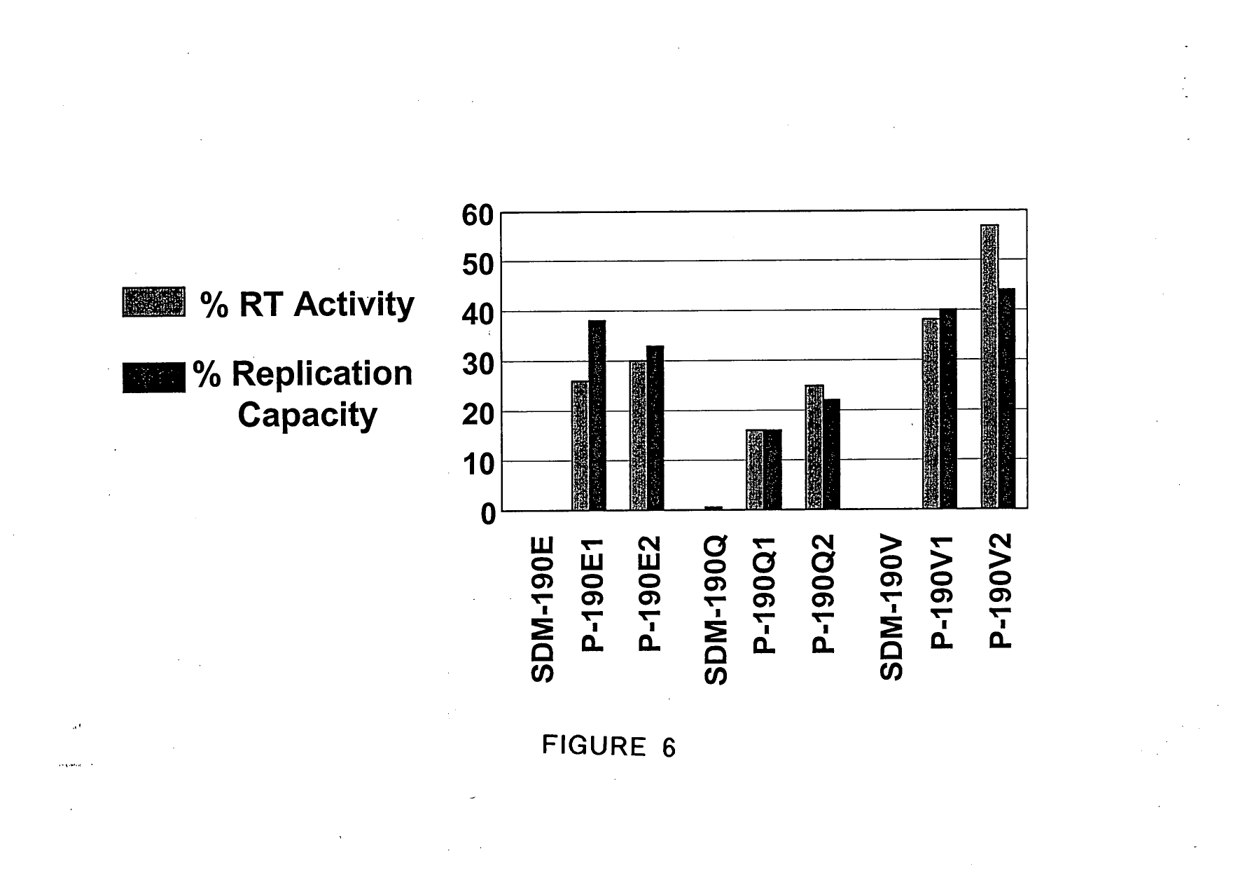

The amount of luciferase activity detected in the infected cells is used as a direct measure of “infectivity,” “replication capacity” or “replication fitness,” i.e., the ability of the virus to complete a single round of replication. Relative fitness is assessed by comparing the amount of luciferase activity produced by patient derived viruses to the amount of luciferase activity produced by a well-characterized reference virus (wildtype) derived from a molecular clone of HIV-1, for example NL4-3 or HXB2. Fitness measurements are expressed as a percent of the reference, for example 25%, 50%, 75%, 100% or 125% of reference (FIGS. 2, 3, 5, 6 and 7).

Host cells were seeded in 10-cm-diameter dishes and were transfected one day after plating with fitness test vector plasmid DNA and the envelope expression vector. Transfections were performed using a calcium-phosphate co-precipitation procedure. The cell culture media containing the DNA precipitate was replaced with fresh medium, from one to 24 hours, after transfection. Cell culture medium containing fitness test vector viral particles was harvested one to four days after transfection and was passed through a 0.45-mm filter before being stored at −80° C. HUV capsid protein (p24) levels in the harvested cell culture media were determined by an EIA method as described by the manufacturer (SIAC; Frederick, Md.). Before infection, target cells (293 and 293/T) were plated in cell culture media. Control infections were performed using cell culture media from mock transfections (no DNA) or transfections containing the fitness test vector plasmid DNA without the envelope expression plasmid. One to three or more days after infection the media was removed and cell lysis buffer (Promega) was added to each well. Cell lysates were assayed for luciferase activity. Alternatively, cells were lysed and luciferase was measured by adding Steady-Glo (Promega) reagent directly to each well without aspirating the culture media from the well.

### 6.2 Example 2

### Measuring Replication Fitness of Viruses with Deficiencies in RT Activity

This example provides methods and compositions for identifying mutations in reverse transcriptase that alter replication fitness. The methods for identifying mutations that alter replication fitness can be adapted to other components of HIV-1 replication, including, but not limited to, integration, virus assembly, and virus attachment and entry. This example also provides a method for quantifying the affect that specific mutations reverse transcriptase have on replication fitness. Means and methods for quantifying the effect that specific protease and reverse transcriptase mutations have on replication fitness can be adapted to mutations in other viral genes involved in HIV-1 replication, including, but not limited to the gag, pol, and envelope genes.

Fitness test vectors were constructed as described in Example 1. Fitness test vectors derived from patient samples or clones derived from the fitness test vector pools, or fitness test vectors engineered by site directed mutagenesis to contain specific mutations, were tested in a fitness assay to determine accurately and quantitatively the relative fitness compared to a well-characterized reference standard. A patient sample was further examined for increased or decreased reverse transcriptase activity correlated with the relative fitness observed.

Reverse transcriptase activity of patient HIV samples: Reverse transcriptase activity can be measured by any number of widely used assay procedures, including but not limited to homopolymeric extension using (e.g. oligo dT:poly rC) or real time PCR based on molecular beacons or 5′ exonuclease activity (Lie and Petropoulos, 1998, Curr Opin Biotechnol. 9(1):43-48). In one embodiment of the invention, the fitness of the patient virus was compared to a reference virus to determine the relative fitness compared to “wildtype” viruses that have not been exposed to reverse transcriptase inhibitor drugs. In another embodiment, the fitness of the patient virus is compared to viruses collected from the same patient at different timepoints, for example prior to initiating therapy, before or after changes in drug treatment, or before or after changes in virologic (RNA copy number), immunologic (CD4 T-cells), or clinical (opportunistic infection) markers of disease progression.

Genotypic analysis of patient HIV samples: Fitness test vector DNAs, either pools or clones, can be analyzed by any genotyping method, e.g., as described above. In one embodiment of the invention, patient HIV sample sequences were determined using viral RNA purification, RT/PCR and ABI chain terminator automated sequencing. The sequence that was determined is compared to reference sequences present in the database or is compared to a sample from the patient prior to initiation of therapy, if available. The genotype was examined for sequences that were different from the reference or pre-treatment sequence and correlated to the observed fitness.

Fitness analysis of site directed mutants: Genotypic changes that are observed to correlate with changes in fitness were evaluated by construction of fitness vectors containing the specific mutation on a defined, wild-type (drug susceptible) genetic background. Mutations were incorporated alone and/or in combination with other mutations that were thought to modulate the fitness of a virus. Mutations were introduced into the fitness test vector through any of the widely known methods for site-directed mutagenesis. In one embodiment of this invention the mega-primer PCR method for site-directed mutagenesis was used. A fitness test vector containing the specific mutation or group of mutations were then tested using the fitness assay described in Example 1 and the fitness was compared to that of a genetically defined wild-type (drug susceptible) fitness test vector that lacked the specific mutations. Observed changes in fitness can be attributed to the specific mutations introduced into the resistance test vector.

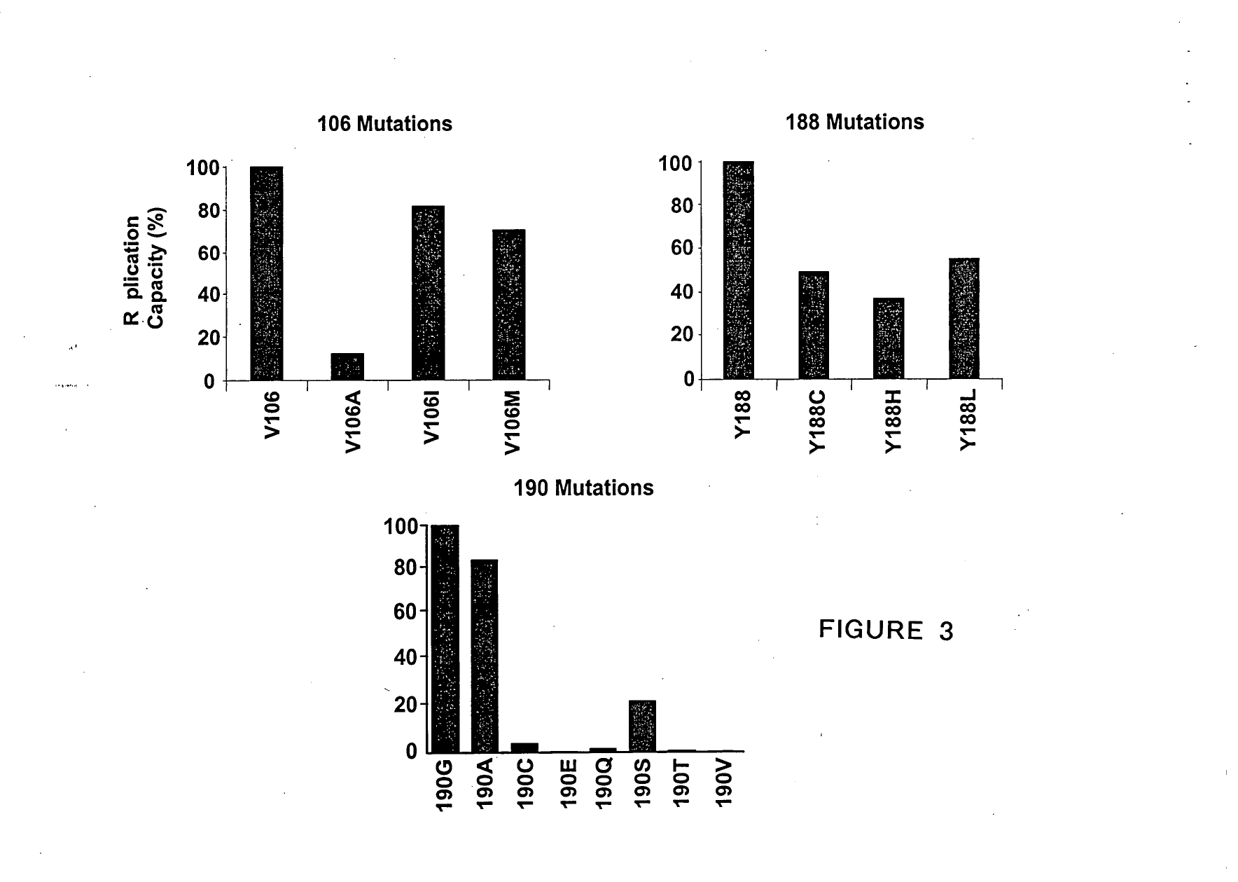

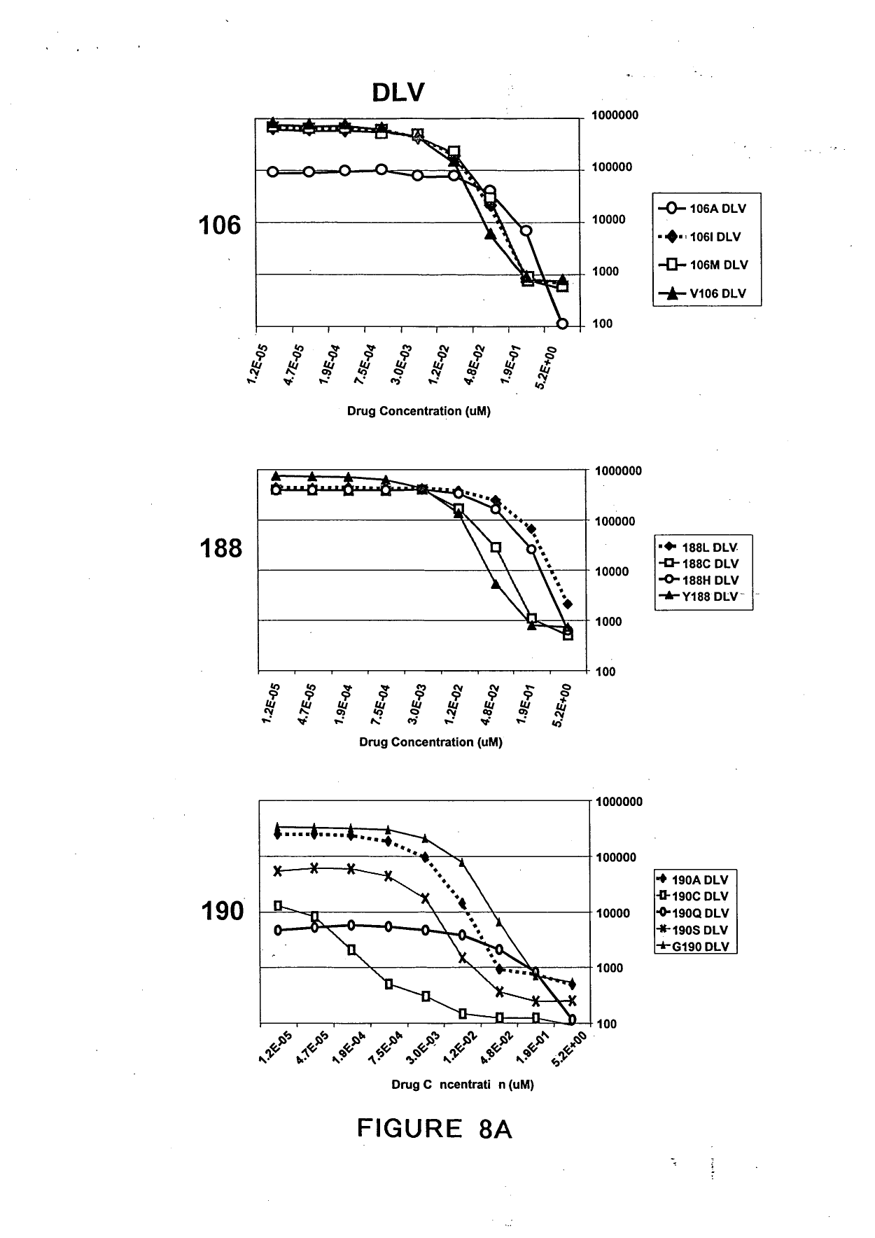

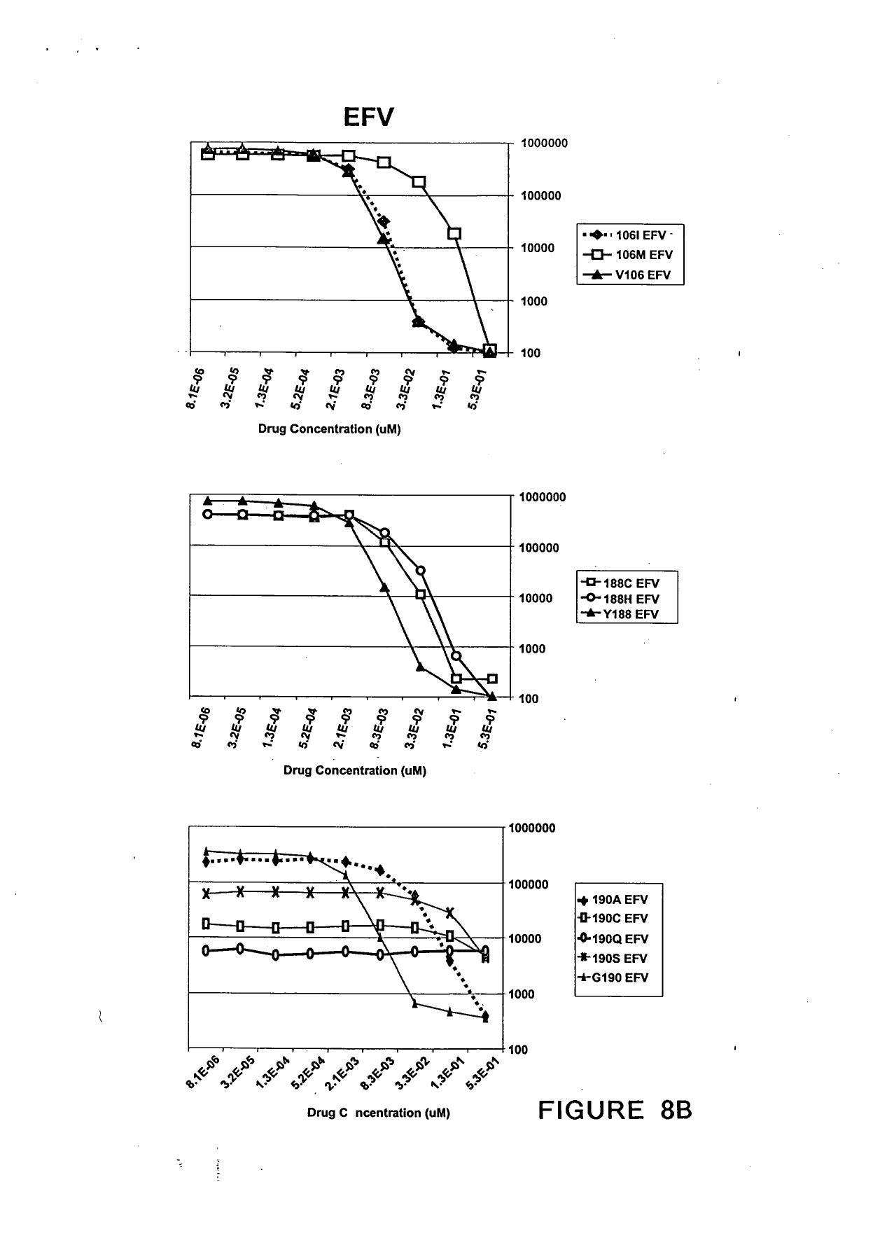

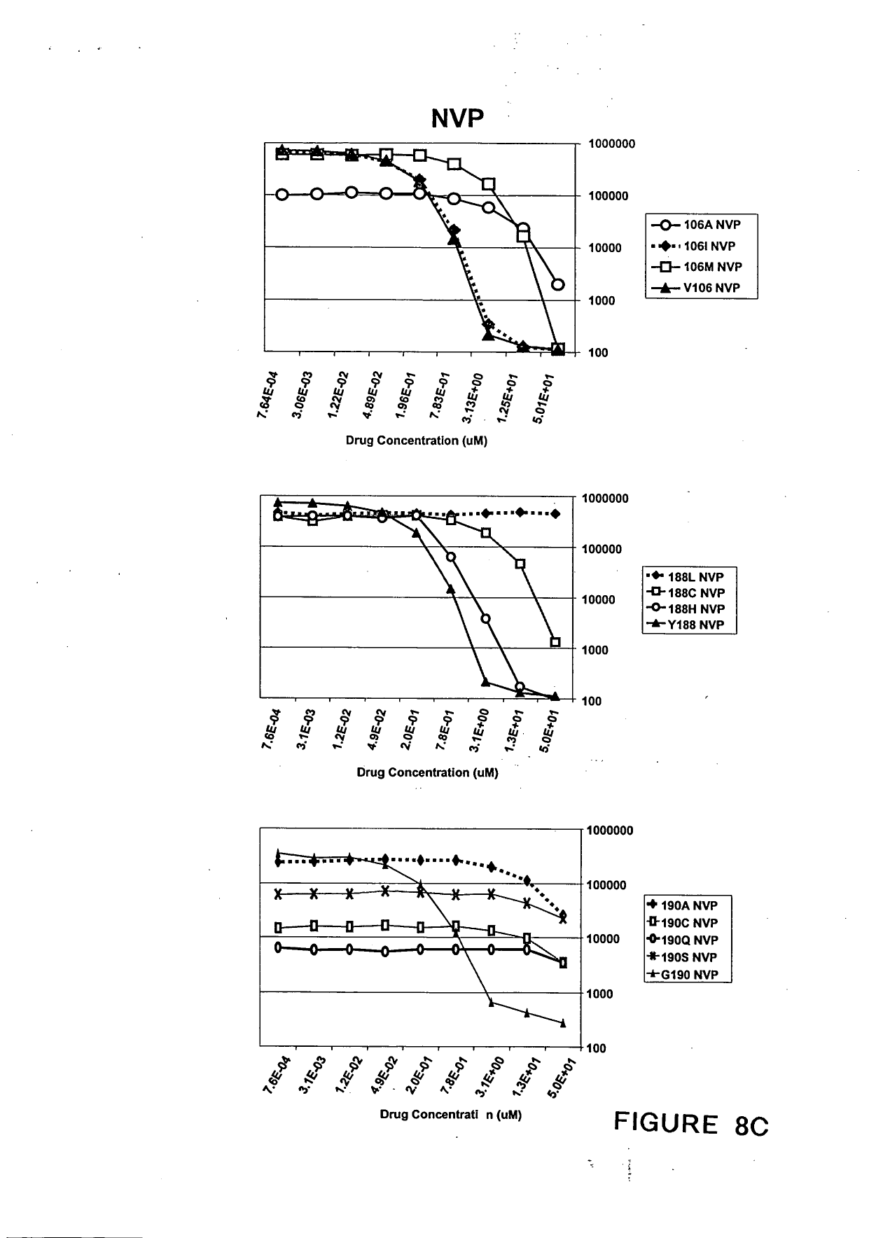

In several related embodiments of the invention, fitness test vectors containing site directed mutations in reverse transcriptase that resulted in amino acid substitutions at position 98 (A98G), 100 (L100I), 101 (K101E), 103 (K103N), 106 (V106A, V106I, V106M), 181 (Y181C, Y188A, Y188C, Y188H, Y188L), or 190 (G190A, G190S, G190C, G190E, G190V, G190T, G190Q) and that displayed different amounts of reverse transcriptase activity were constructed and tested for fitness (FIGS. 2, 3, 4, 5, 6, 7 and 8A-8C). The fitness results established a correlation between specific reverse transcriptase amino acid substitutions and fitness. The data demonstrated that different mutations at the same position can have drastically different effects on replication capacity. FIG. 3 shows that V106I and V106M had relatively high replication capacities compared to V106A. The differences observed between different mutations at position 190 were particularly striking, ranging from barely detectable to greater than 80% of wild-type. These differences in replication capacity also were manifest when the mutant viral strains are replicated in the presence of NNRTIs, as shown in FIGS. 8A-8C.

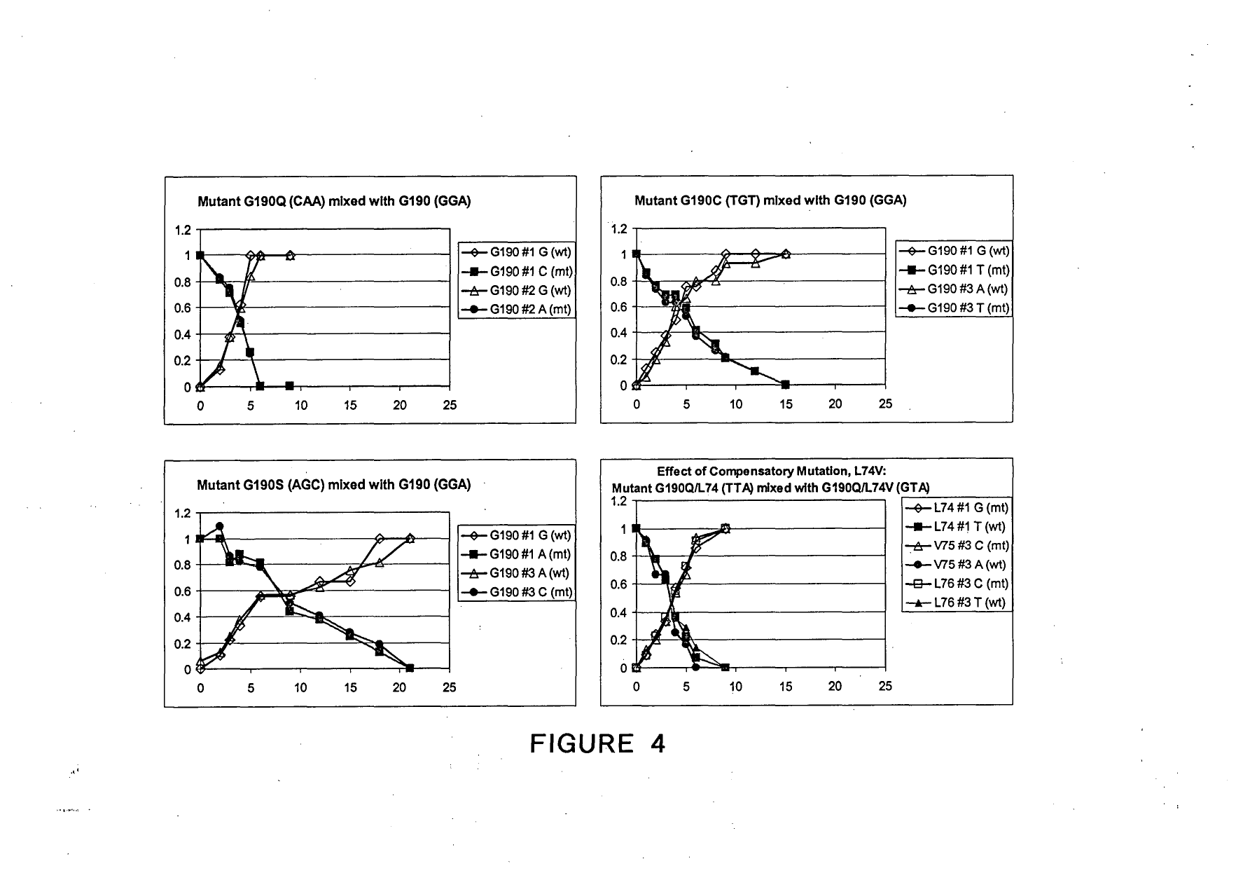

FIG. 4 demonstrates that the replication capacity measurements performed as described above produced results that were consistent with measurements made using replication competition assay results.

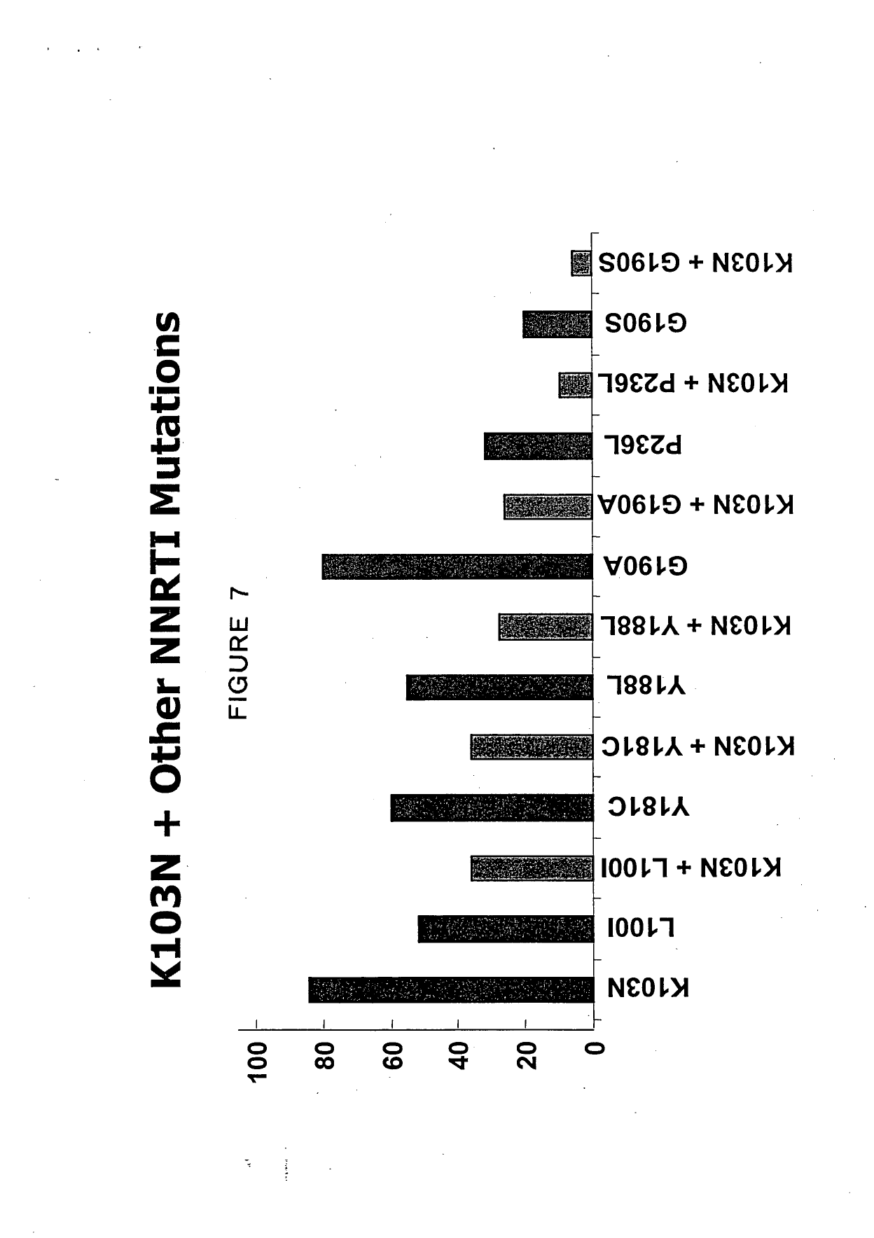

Viruses containing multiple mutations also were constructed and tested as shown in FIG. 2. FIG. 7 compares the replication capacities of single mutant strains and double-mutant strains comprising combinations of the single mutations and shows that strains having combinations of mutations had replication capacities less than strains having either mutation alone.

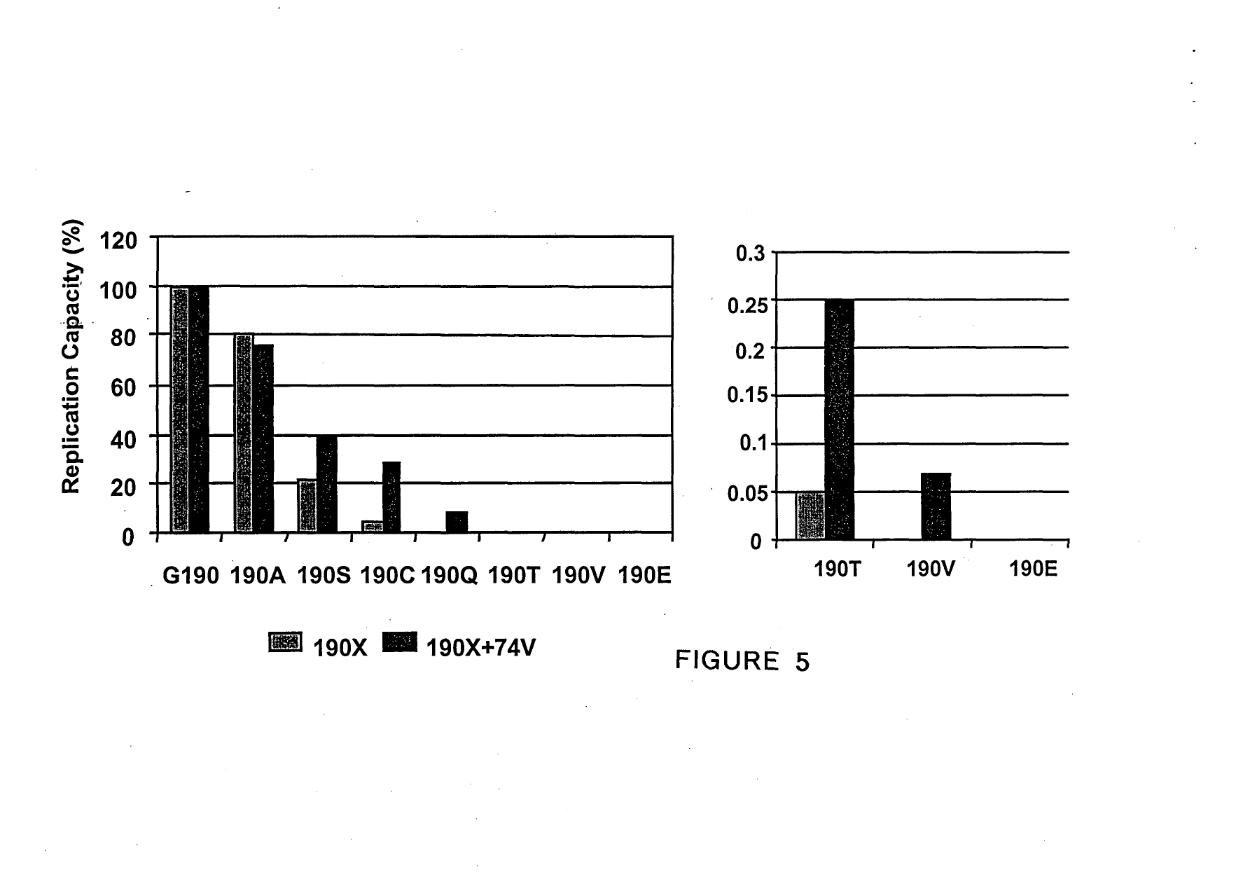

Mutant strains comprising both a mutation at position 190 and the mutation L74V were also constructed as shown in FIG. 5. While the L74V mutation did not affect the replication capacity of a strain that was wild-type at position 190, it did increase the replication capacity of most other position 190 mutations.

### 6.3 Example 3

### Analysis of Patient Samples to Identify Resistance-Associated Mutations

This example demonstrates a method of analyzing patient samples so as to identify mutations that are associated with NNRTI resistance. It also demonstrates that K101P, K103R and V179D as well as the combination of K103R and V179D are new NNRTI-resistance mutations.

In order to determine the relationship between an HIV-1 strain's reverse transcriptase (RT) sequence and its susceptibility to NNRTIs, a data set of 18,034 samples was analyzed genotypically as well as phenotypically. Those samples with NNRTI resistance in the absence of well-characterized NNRTI mutations were identified by univariate and combinatorial correlation analysis. 8,673 samples had no mutation at position 100, 181, 188, 190 or 227, nor any of the following mutations in RT: A98G, K101E, K103N/S, V106A/M, P225H, M230L, or P236L, but still had high-level reductions in susceptibility to one or more NNRTIs. Of the 8,673 samples, 146 samples exhibited >5-fold reduction in susceptibility to at least one NNRTI.

All amino acid positions of RT where at least two samples had a mutation in an unmixed form (i.e., no mixture of mutation and wild type) were identified. The amino acid positions and residues identified were: P4, E6, K20, T27, V35, T39, M41, K43, E44, S48, K49, I50, V60, A62, K64, D67, S68, T69, K70, L74, V75, F77, R83, D86, V90, 194, A98, K100, Q102, K103, K104, V106, V108, Y115, F116, V118, D121, K122, D123, I135, E138, T139, I142, Q151, A158, C162, T165, K166, I167, E169, K173, Q174, P176, D177, I178, V179, M184, V189, E194, G196, Q197, T200, I202, E203, Q207, H208, L210, R211, F214, T215, D218, K219, H221, L228, D237, K238, P243, V245, E248, D250, I257, A272, K275, V276, R277, Q278, K281, L283, R284, T286, A288, E291, V292, V293, P294 and E297.

The amino acids present at each of the above positions were then determined and any amino acid present in more than one sample was analyzed separately. Correlation analyses were performed using Statview software (SAS Institute, Cary, N.C., USA) to determine amino acid mutations correlated with each NNRTI continuous fold change (FC), or with a dichotomous fold change (FC>10).

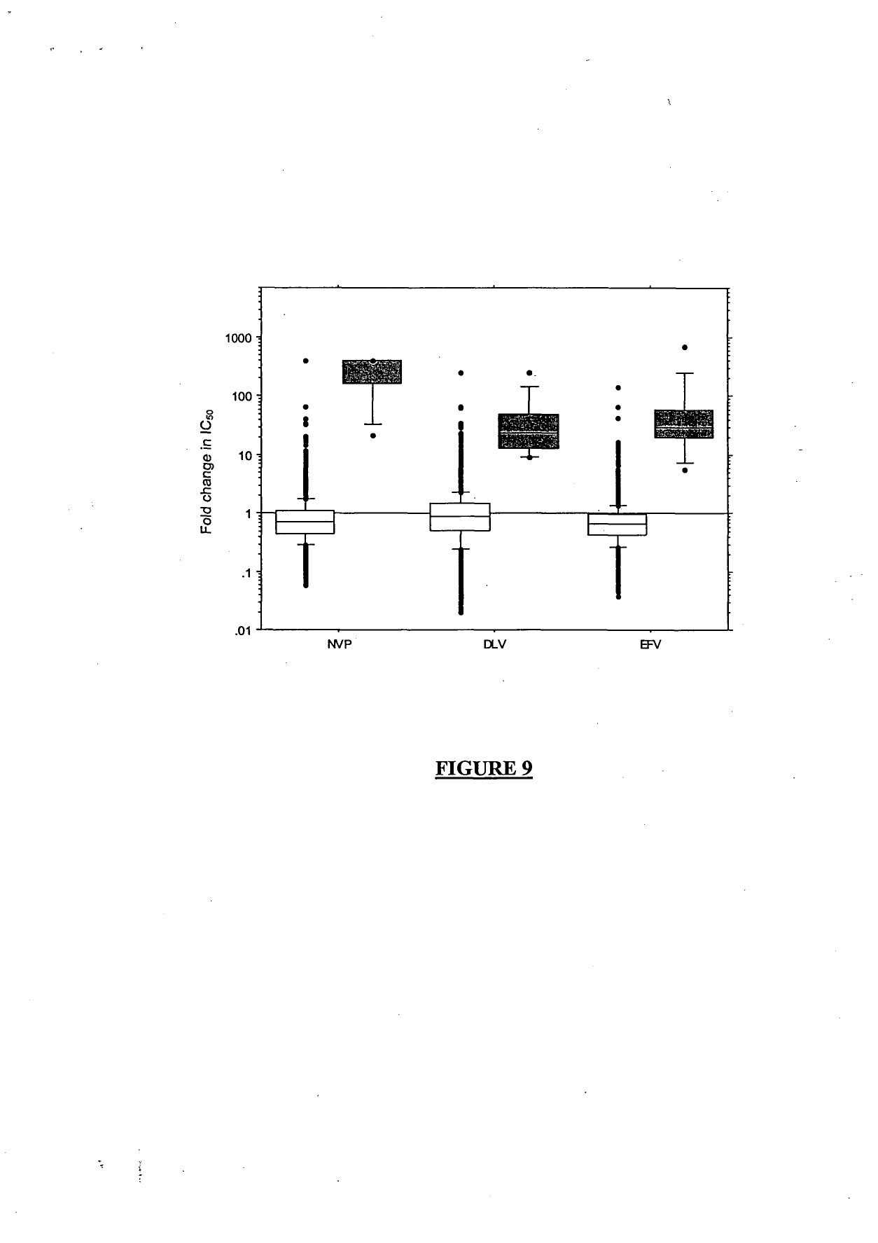

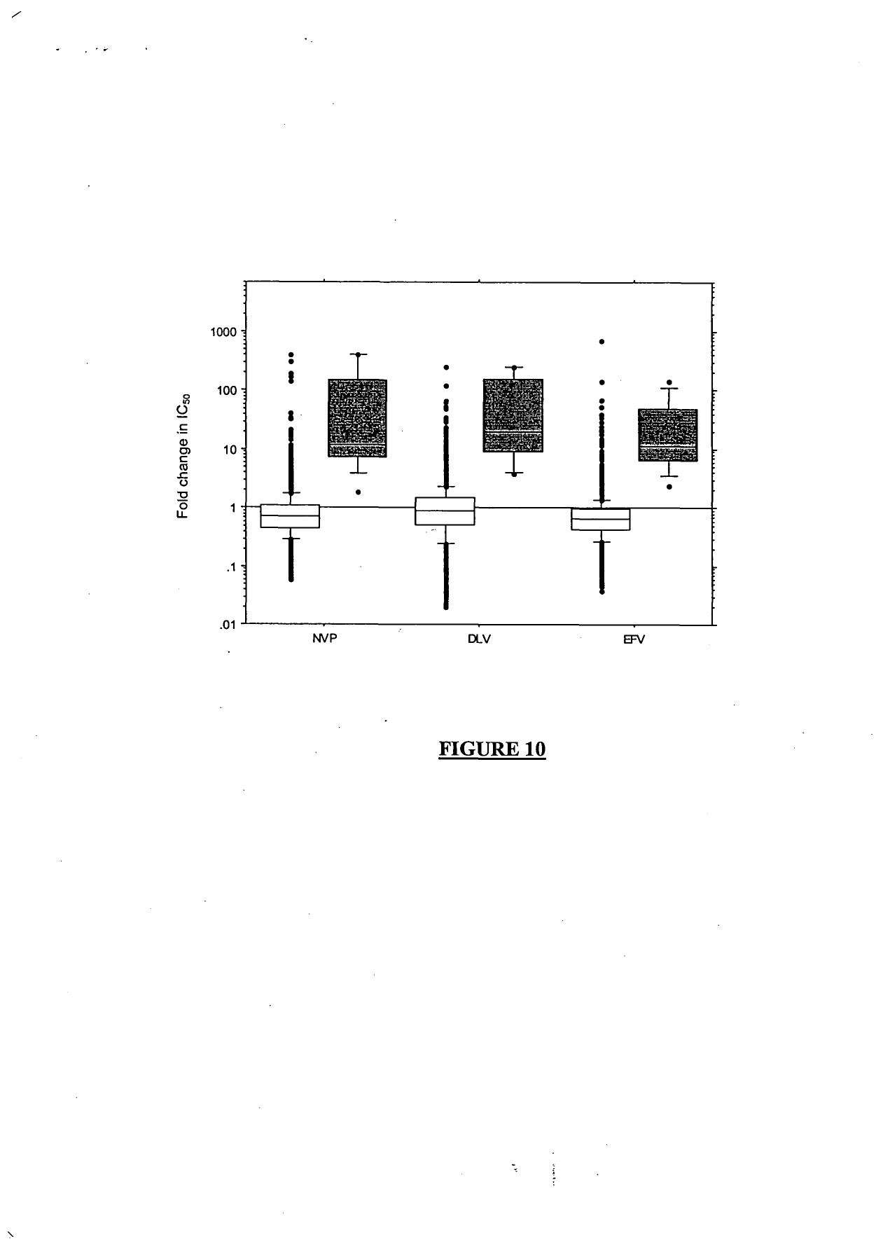

The data (RT mutations, FC for nevirapine (NVP), delavirdine (DLV) and efavirenz (EFV)) for the samples that were most resistant to NNRTIs from amongst the 146 samples analyzed are shown in Table 1. Table 1 provides the RT genotypes and NNRTI FC in susceptibility observed in samples that exhibited a FC greater than ten with at least one NNRTI. The maximum FC values observed for NVP, DLV and EFV were >400-, >250- and >144-fold, respectively. As illustrated in FIGS. 9 and 10 and Table 1, samples with the highest FC contained a proline substitution at position 101 (n=10, median NVP, DLV and EFV fold change was 355-, 26- and 26-fold, respectively) or a combination of 103R and 179D (n=13, median NVP, DLV and EFV fold change was 12-, 24- and 17-fold, respectively). The presence of K101P, K103R and V179D in the RT genotypes are indicated in bold in column 1 of Table 1.

All of the 101P samples, and all but 2 of the 103R/179D samples, also had mutations associated with nucleoside RT inhibitor (NRTI) resistance. In samples without 101P or 103R/179D, the maximum FC for NVP, DLV and EFV was 41-, 67- and 15-fold, respectively. Mutations associated with reduced susceptibility to all three NNRTIs independent of 101P, 103R/179D, or NRTI-associated mutations, included 101Q, 106, I135T, 166R, 179D, 189I, 245T, 272S, and 297T. In some samples, surprising correlations with decreased resistance or increased sensitivity, and possibly hypersusceptibility to NNRTIs was also seen at positions 245 and 138.

Correlation of K101P, K103R/V179D and other mutations identified as NNRTI-resistance mutations to a decreased susceptibility to NNRTIs was verified by analyzing samples without NNRTI resistance. A total of 526 samples without NNRTI resistance were analyzed. The NNRTI fold change of these samples were all between 1 and 2, indicating no NNRTI resistance. Genotypic analysis of the 526 samples showed that none of the mutations identified as NNRTI-resistance mutations were present in any of the samples, confirming that the NNRTI-resistance mutations were indeed associated with a decreased susceptibility to NNRTIs.

This example demonstrates a method for determining whether a human immunodeficiency virus (HIV), e.g., human immunodeficiency virus type 1 (HIV-1), has an increased likelihood of having resistance to treatment with a NNRTI, comprising: detecting whether the reverse transcriptase (RT) encoded by said HIV-1 exhibits the presence or absence of a mutation associated with resistance to treatment with said NNRTI at amino acid position 101, 103 or 179 of an amino acid sequence of said RT, e.g., K101P, K103R or V179D, wherein the presence of said mutation indicates that the HIV-1has an increased likelihood of having resistance to treatment with the NNRTI. In general, the methods can comprise detecting the presence or absence of any combinations of mutations listed herein associated with NNRTI resistance. For example, the method can comprise detecting the presence or absence of a mutation at 2 or all 3 amino acid positions associated with NNRTI resistance. In certain embodiments, such a method can comprise detecting the presence or absence of K103R and V179D. Examples of NNRTIs include, but are not limited to, delavirdine, nevirapine and efavirenz.

All references cited herein are incorporated by reference in their entireties.

The examples provided herein, both actual and prophetic, are merely embodiments of the present invention and are not intended to limit the invention in any way.

TABLE 1
RT Genotypes and NNRTI Fold-Change in Susceptibility Data for Samples
With At Least One NNRTI FC Greater Than 10

| RT MUTATIONS IN SAMPLE | NVP FC* | DLV FC* | EFV FC |
|---|---|---|---|
| A62A/V**, K101P, Q102K, D123E, I135T, T139V, C162S, I178M, M184V, G196E, R211G, F214L, | 400.0 | 121.1 | 70.3 |
| H221H/Y, L228L/R, A272P, R277K, K281R, T286P, E297K | | | |
| K20R, M41L, K43E, E44D, D67N, L74I, A98S, K101P, Q102K, V118I, D123E, C162S, D177E, | 400.0 | 23.4 | 20.6 |
| I178I/L, M184V, G196G/E, E203E/K, Q207E, H208Y, R211K, T215Y, D218E, K219Q, L228H, | | | |
| V245E, R277K, T286A, E297K | | | |
| M41L, K43K/N, L74L/V, V75V/L, K101P, Q102K, V108I, V118V/I, I135T, C162S, V179L, | 400.0 | 19.6 | 700.0 |
| M184V, L210W, R211K, T215Y, L228R, V245E, R277K, T286T/A, E297R, L301I | | | |
| K64K/N, D67N, T69T/N, K70R, K101P, C162S, I178M, R211K; K219Q, H221Y, K238T, V245E, | 400.0 | 250.0 | 144.6 |
| R277K | | | |
| M41L, K101P, Q102K, C162S, M184V, T215Y, H221Y, V245T, V293I, E297K | 311.5 | 48.5 | 40.1 |
| V35R, T39A, T69D, K82R, R83K, K101P, Q102K, K122E, I135T, I142I/T, Q151M, C162Y, | 196.8 | 29.6 | 29.3 |
| K173E, V179V/I, T215Y, I244I/V, A272P, R277K, E297K | | | |
| K13K/N, M41L, K43Q, E44D, D67N, L74V, I94L, K101P, Q102K, V118I, K122E, D123E, I135T, | 172.9 | 35.4 | 22.0 |
| N137N/S, C162S, M184V, T200A, E203K, Q207E, L210W, T215Y, D218E, K219N, K223Q, | | | |
| L228H, Q242H, R277K, T286T/A | | | |
| V35I, E44E/D, K49E, I50I/T, A62V, Q102K/R, K103R, Y115F, V118I, I135I/T, E138A, I142R, | 400.0 | 250.0 | 67.3 |
| C162S, I178L, V179D, M184V, F214L, T215Y, D218D/E, H221H/Y, A272P, V276I, R277K, L283I | | | |
| V35V/E, K64H, D67N, Q102K, K103R, V118I, I135T, T139R, C162S, T165L, V179D, M184V, | 400.0 | 250.0 | 142.5 |
| F214L, T215F, K219Q, V245M, R277K, A288S | | | |
| L34I, M41L, K64K/R, Q102K, K103R, D121Y, K122E, D123E, I178M, V179D, M184V, G196E, | 65.7 | 64.4 | 42.5 |
| T200I, R211G, T215Y, V245M, A272P, R277K, V293I, P294Q | | | |
| K64K/R, Q102K, K103K/R, V118I, K122A, D123E, C162S, E169D, V179D, T200A, F214L, | 19.1 | 33.2 | 18.0 |
| V254V/I, A272P, R277K, T286A, A288A/S, V293I | | | |
| T69T/N, R83K, V90I, Q102K, K103K/R, K122E, C162S, V179D, G196E, T200T/A/I/L/S/V, | 14.6 | 26.4 | 19.4 |
| T215T/S, V245V/M, A272P, R277K | | | |
| E6E/K, A62A/V, K64R, K70R, Q102K, K103R, K122E, C162S, I178M, V179D, M184V, | 11.6 | 22.6 | 10.9 |
| T215T/F/I/S, S251S/I, T286T/A | | | |
| Q102K, K103K/R, V108I, D123E, C162S, K173K/E, D177D/E, V179V/D, Q207E, R211K, E248V, | 11.3 | 15.6 | 11.6 |
| A272P, R277R/K, V292I | | | |
| Q102K, K103K/R, V106I, D123D/E, I135I/V, C162S, E169D, K173R, Q174K, V179D, M184V, | 10.9 | 57.7 | 21.0 |
| Q207K, R211A, K219R, A272P, R277K, V293I | | | |
| V35M, A62A/V, D86E, Q102K, K103R, D123E, C162S, D177K, V179D, M184V, I202V, Q207A, | 10.0 | 17.5 | 11.4 |
| R211K, V241I, R277K, A288G | | | |
| A98S, Q102K, V106I, T107A, K122E, I135T, C162S, K166R, K173S, Q207E, A272S, R277K, | 34.0 | 34.8 | 15.3 |
| T286A, V292I, V293I | | | |
| P4S, L74V, Q102K, Y115F, K122K/E, C162S, Q174K, I178M, M184V, K201K/R, I202I/V, Q207E, | 19.2 | 67.3 | 13.1 |
| R211R/A/G/T, T240T/S, A272P, R277K, T286A, V293I | | | |
| Q102K, I135T, C162S, K173E, I178L, V179D, G196E, R211S, F214L, K220K/E, D237E, A272P, | 16.5 | 11.5 | 14.2 |
| R277K, E297T | | | |
| V35V/I, T39A, M41L, K43K/D/E/N, E44D, V60V/I, D67N, K101P, Q102K, V106I, V118I, | 400.0 | 9.1 | 24.2 |
| D121D/H, K122K/E, D123D/E/K/N, C162S, K166K/R, V179I, G196E, T200A, E203D, H208F, | | | |
| L210W, R211K, T215Y, D218E, K219N, D250E, A272P, 1274I/V, R277R/K, A288S, E297K | | | |
| K20R, M41L, K43E, E44A, D67S, T69SCT, L74V, R83K, A98S, K101P, Q102K, V118I, K122E, | 142.3 | 9.3 | 18.2 |
| D123E, I135T, C162D/G, K166R, D177D/N, I178M, V179V/I, G196E, T200A, L210W, R211K, | | | |
| T215Y, V245E, A272P, R277K, K281K/R, V293I, E297K | | | |
| V35I, A62V, D67G, T69G, K70K/R, V75I, F77L, R83K, K101P, Q102K, K104K/R, F116Y, K122E, | 35.4 | 22.4 | 7.8 |
| D123N, Q151M, M184V, I202V, Q207N, K219E, A272P, R277K, T286A, V293I, E297A | | | |
| K20R, K49R, N81N/S, A98S, Q102K, K122E, A158S, C162S, T165L, E169D, I178M, M184M/V, | 40.6 | 29.1 | 5.5 |
| G196E, T200T/I, V245E, V276I, R277K, T296S | | | |
| I31L, R83R/K, V90V/I, K101K/Q, Q102K, V118V/I, K122E, D123N, C162S, V179V/I, M184V, | 20.3 | 21.2 | 7.0 |
| L210L/S, R211K, H221H/Y, L228L/R, R277K, V293I | | | |
| V60I, Q102K, I135T, I142V, C162S, T165L, M184V, T200A, Q207E, R211K, A272S, L283I, | 17.8 | 17.2 | 6.8 |
| T286A, E297K | | | |
| E28A, K70R, K101N, Q102K, C162S, Q207E, R277K, V293I | 10.3 | 21.9 | 8.1 |
| D67N, T69D, K70R, Q102K, K103R, K122E, D123E, I135T, C162S, V179E, F214L, K219Q, | 5.9 | 22.9 | 11.8 |
| V245E, A272P, R277K, K281R, A288T, V293V/I, E297K | | | |
| K20R, K32K/R, M41L, K43K/E, D67D/G, S68S/G, L74I, V75V/A, Q102K, K103R, V118V/I, | 19.9 | No Data | 16.7 |
| I135M, C162S, K166K/T, I178I/M, V179D, M184V, V189I, T200A, H208H/Y, L210W, R211K, | | | |
| T215Y, R277K, R284K, T286A, V293I | | | |
| V21I, Q102K, T139R, C162S, M184V, T200A, Q207E, R211K, K238N, A272P, R277K, V293I, | 34.1 | 18.7 | 4.0 |
| P294T, E297K | | | |
| Q102K, I135T, C162S, K166R, K173Q, Q207E, R211K, V245T, A272P, V293I, E297R | 20.3 | 14.7 | 4.4 |
| K20R, K49R, D67N, T69N, K70K/R, K101H, Q102K, D123E, I135T, C162S, D177E, M184V, | 20.1 | 64.1 | 4.6 |
| V189V/I, I195I/V, G196E, E203K, K219Q, R277K, K281R, L283I, V293I, E297K, L303L/R | | | |
| K49R, V90I, Q102K, C162D, K166R, D177E, I178M, V245M, A272P, R277K, V293I | 11.6 | 14.6 | 2.4 |
| S3S/C, V35K, T39A, M41L, K43E, D67H, S68N, T69N, K101H, Q102K, K122E, D123E, I135T, | 10.6 | 23.3 | 1.7 |
| I142V, A158A/S, C162C/Y, Q174E/K, N175N/Y, G196G/E, Q207S, T215Y, K219N, A272P, | | | |
| R277K, A288A/T, V293I, E297T | | | |
| K20R, V35V/I, Q102K, K103R, K122E, I135V, C162S, E169D, Q174K, V179D, M184V, G196E, | 6.9 | 14.3 | 6.8 |
| T200A, F214L, V245E, R277K, T286A, A288S | | | |
| K101K/Q, Q102K, K122K/E, I135L/V, C162S, K173K/T, I178L, M184V, R211K, A272P, L283I | 9.1 | 10.1 | 5.2 |
| E6D, R83K, K101Q, Q102K, I135I/L/S/T, E138E/A, C162S, K173R, L210F, R211K, K275K/Q, | 8.8 | 23.0 | 6.9 |
| V276T | | | |
| R83K, Q102K, C162S, E169D, I178L; V179E, I202I/V, R211K, A272P, R277K, V293I | 6.9 | 16.4 | 9.4 |
| E6D, M41L, K49R, V60I, Q102K, I135I/T, E138E/D, C162S, K166I, I178M, V179I, T200T/A, | 33.0 | 7.1 | 4.3 |
| E203D, L210L/W, R211R/K, T215Y, L228L/R, A272P, V293I, E297K | | | |
| E6D, V35V/I, A98A/S, Q102K, I135T, C162S, K173E, T200T/A, Q207E, R211R/K, K238T, | 14.7 | 7.6 | 4.8 |
| V245M, S251S/I, A272P, K275R, R277K, T286A, E297K | | | |
| V35V/L, K49R, A98S, Q102K, K122K/E, A158S, C162S, E169D, Q174Q/K/R, I178I/L/M, T200A, | 9.6 | 11.3 | 3.2 |
| Q207E, M230M/V, I257L, V261I, R277K | | | |
| Q102K, D121Y, K122E, I135I/T, I142I/T, C162S, I178I/L, V189I, R211K, V245T, R277K, E297T | 8.7 | 12.0 | 4.3 |
| V60V/I, D67N, K70R, V90I, Q102K, V106V/I, T139K, I142V, C162C/Y, E169D, R211K, K219Q, | 8.0 | 32.2 | 2.2 |
| V245E, A272P, A288S, P294Q | | | |
| K49R, Q102K, I135T, C162S, Q207E, V245M, R277K, T286A, V293I | 6.6 | 10.7 | 2.5 |
| A98S, Q102K, D121Y, K122E, I135L, E138A, C162S, V179I, M184M/V, Q207E, R211K, P243S, | 6.0 | 20.2 | 2.6 |
| V245E, R277K, E297A | | | |
| Q102K, C162S, I178M, V179E, T200A, Q207R, R277K, A288T | 4.5 | 12.5 | 6.7 |
| K20R, Q102K, I135L, V179D, T200A, Q207N, R211K, T286A, V293I, P294T | 2.9 | 10.6 | 5.2 |
| K64H, D67N, T69N, K70R, V90I, Q102K, C162S, P176Q, G196E, T200A, K219Q, L228H, K238T, | 4.1 | 10.2 | 2.0 |
| V245E, A272P, R277K, Q278H, T286P, E297R | | | |
| P4P/S, V35T, T39K/R, S48T, Q102K, K122E, E138A, K173T, D177E, V179D, T200A, I202I/V, | 3.0 | 10.4 | 2.1 |
| Q207E, F214L, V245Q, E248D/N, A272P, K275Q, R277R/K, L283L/I, T286T/A, E291D, V292I, | | | |
| P294P/Q | | | |

*The maximum detectable FC for NVP was 400 and for DLV was 250.
**The presence of a mixture of amino acids at the listed positions in any sample is indicated by listing the amino acids observed, separated by a slash (“/”).

What is claimed is:

1. A method for determining whether a human immunodeficiency virus type 1 (“HIV-1”) has an increased likelihood of having an impaired replication capacity, comprising: detecting whether the reverse transcriptase encoded by said HIV-1 exhibits the presence or absence of a mutation associated with impaired replication capacity at amino acid position 98, 100, 101, 103, 106, 108, 179, 181, 188, 190, 225 or 236 of the amino acid sequence of said reverse transcriptase, wherein the presence of said mutation indicates that the HIV-1 has an increased likelihood of having impaired replication capacity, with the proviso that said mutation is not P236L.

2. The method of claim 1, wherein the mutation associated with impaired replication capacity is selected from the group consisting of A98G, L100I, K101E, K103N, V106A, V106I, V106M, Y181C, Y188A, Y188C, Y188H, Y188L, G190A, G190C, G190E, G190T, G190V, G190Q, G190S and G190V.

3. The method of claim 1, wherein said mutation confers resistance to a non-nucleoside reverse transcriptase inhibitor.

4. The method of claim 3 wherein said non-nucleoside reverse transcriptase inhibitor is nevirapine, delavirdine or efavirenz.

5. A method for determining whether a subject has an HIV-1 with an increased likelihood of having an impaired replication capacity, comprising: detecting whether the reverse transcriptase encoded by said HIV-1 exhibits the presence or absence of a mutation associated with impaired replication capacity at amino acid position 98, 100, 101, 103, 106, 108, 179, 181, 188, 190, 225 or 236 of the amino acid sequence of said reverse transcriptase, wherein the presence of said mutation indicates that the HIV-1 has an increased likelihood of having impaired replication capacity, with the proviso that said mutation is not P236L.

6. The method of claim 5, wherein the mutation associated with impaired replication capacity is selected from the group consisting of A98G, L100I, K101E, K103N, V106A, V106I, V106M, Y181C, Y188A, Y188C, Y188H, Y188L, G190A, G190C, G190E, G190T, G190V, G190Q, G190S and G190V.

7. The method of claim 5, wherein said mutation confers resistance to a non-nucleoside reverse transcriptase inhibitor.

8. The method of claim 7, wherein said non-nucleoside reverse transcriptase inhibitor is nevirapine, delavirdine or efavirenz.

9. The method of claim 5, wherein the subject is undergoing or has undergone prior treatment with an antiviral drug.

10. The method of claim 1, wherein the method comprises detecting the presence or absence of a mutation associated with impaired replication capacity at at least 2, 3, 4, 5, 6, 7, 8, 9, 10, 11 or 12 amino acid positions.

11. The method of claim 10, wherein the method comprises detecting the presence or absence of a mutation associated with impaired replication capacity at amino acid positions 106 and 181; 103 and 190; 103 and 236; 181 and 236; 103 and 188; 103 and 181; 100 and 103; or 98 and 181.

12. The method of claim 10, wherein the method comprises detecting the presence or absence of a mutation associated with impaired replication capacity selected from the group consisting of: V106A and Y181C; K103N and G109S; P236L and K103N; P236L and Y181C; K103N and G190A; K103N and Y181C; K103N and Y188L; L100 I and K103N; and Y181C and A98G.

13. The method of claim 10, wherein the method comprises detecting the presence or absence of a mutation associated with impaired replication capacity at amino acid positions 103, 181 and 236; 100, 103, and 190; or 103, 181 and 225.

14. The method of claim 10, wherein the method comprises detecting the presence or absence of a mutation associated with impaired replication capacity selected from the group consisting of: P236L, K103N and Y181C; L100I, K103N and G190S; and K103N, Y181C and P225H.

15. The method of claim 2, wherein the method comprises detecting the presence or absence of a mutation associated with impaired replication capacity at at least 2, 3, 4, 5, 6, 7, 8, 9, 10, 11 or 12 amino acid positions.

16. The method of claim 15, wherein the method comprises detecting the presence or absence of a mutation associated with impaired replication capacity at amino acid positions 106 and 181; 103 and 190; 103 and 236; 181 and 236; 103 and 188; 103 and 181; 100 and 103; or 98 and 181.

17. The method of claim 15, wherein the method comprises detecting the presence or absence of a mutation associated with impaired replication capacity selected from the group consisting of: V106A and Y181C; K103N and G109S; P236L and K103N; P236L and Y181C; K103N and G190A; K103N and Y181C; K103N and Y188L; L100I and K103N; and Y181C and A98G.

18. The method of claim 15, wherein the method comprises detecting the presence or absence of a mutation associated with impaired replication capacity at amino acid positions 103, 181 and 236; 100, 103, and 190; or 103, 181 and 225.

19. The method of claim 15, wherein the method comprises detecting the presence or absence of a mutation associated with impaired replication capacity selected from the group consisting of: P236L, K103N and Y181C; L100I, K103N and G190S; and K103N, Y181C and P225H.

20. An isolated oligonucleotide between about 10 and about 40 nucleotides long encoding a portion of a HIV reverse transcriptase in a HIV-1 that comprises at least one mutation at amino acid position 98, 100, 101, 103, 106, 108, 179, 181, 188, 190, 225 or 236 of an amino acid sequence of said reverse transcriptase in said HIV-1, wherein the mutation is associated with reduced susceptibility to a protease inhibitor, with the proviso that said mutation is not P236L.
